SEQUENCE LISTING (14 sequences)

SEQ ID NO: 1 - DNA, 130 bp, Homo sapiens
taatatatttcttcatgaagacctcacagtaaaaataggtgattttggtctagctacagtgaaatctcgatggagtgggtcccatcagtttgaacagttgtctggatccattttgtggatggcaccagaa

SEQ ID NO: 2 - DNA, 18 bp, Artificial Sequence
  Primer
cactccatcgagatttca

SEQ ID NO: 3 - DNA, 19 bp, Artificial Sequence
  Primer
ccactccatcgagatttct

SEQ ID NO: 4 - DNA, 27 bp, Artificial Sequence
  Primer
atatttcttcatgaagacctcacagta

SEQ ID NO: 5 - DNA, 21 bp, Artificial Sequence
  PCR Probe
aggtgattttggtctagctac

SEQ ID NO: 6 - DNA, 18 bp, Artificial Sequence
  Primer
ctggtgcagctccttggg

SEQ ID NO: 7 - DNA, 19 bp, Artificial Sequence
  Primer
ccgtaggtcatgaactcag

SEQ ID NO: 8 - DNA, 19 bp, Artificial Sequence
  Primer
ccgtaggtcatgaactcaa

SEQ ID NO: 9 - DNA, 17 bp, Artificial Sequence
  PCR Probe
ccccgttctatatcatc

SEQ ID NO: 10 - protein, 744 aa, Homo sapiens
MAALSGGGGGGAEPGQALFNGDMEPEAGAGAGAAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLAEDFSLYACASPKTPIQAGGYGAFPVH

SEQ ID NO: 11 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 12 - DNA, 5388 bp, Homo sapiens
aaaatgttggagatctgcctgaagctggtgggctgcaaatccaagaaggggctgtcctcgtcctccagctgttatctggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtagcagcagtcaggggtcaggtgtcaggcccgtcggagctgcctgcagcacatgcgggctcgcccatacccgtgacagtggctgacaagggactagtgagtcagcaccttggcccaggagctctgcgccaggcagagctgagggccctgtggagtccagctctactacctacgtttgcaccgcctgccctcccgcaccttcctcctccccgctccgtctctgtcctcgaattttatctgtggagttcctgctccgtggactgcagtcggcatgccaggacccgccagccccgctcccacctagtgccccagactgagctctccaggccaggtgggaacggctgatgtggactgtctttttcatttttttctctctggagcccctcctcccccggctgggcctccttcttccacttctccaagaatggaagcctgaactgaggccttgtgtgtcaggccctctgcctgcactccctggccttgcccgtcgtgtgctgaagacatgtttcaagaaccgcatttcgggaagggcatgcacgggcatgcacacggctggtcactctgccctctgctgctgcccggggtggggtgcactcgccatttcctcacgtgcaggacagctcttgatttgggtggaaaacagggtgctaaagccaaccagcctttgggtcctgggcaggtgggagctgaaaaggatcgaggcatggggcatgtcctttccatctgtccacatccccagagcccagctcttgctctcttgtgacgtgcactgtgaatcctggcaagaaagcttgagtctcaagggtggcaggtcactgtcactgccgacatccctcccccagcagaatggaggcaggggacaagggaggcagtggctagtggggtgaacagctggtgccaaatagccccagactgggcccaggcaggtctgcaagggcccagagtgaaccgtcctttcacacatctgggtgccctgaaagggcccttcccctcccccactcctctaagacaaagtagattcttacaaggccctttcctttggaacaagacagccttcacttttctgagttcttgaagcatttcaaagccctgcctctgtgtagccgccctgagagagaatagagctgccactgggcacctgcgcacaggtgggaggaaagggcctggccagtcctggtcctggctgcactcttgaactgggcgaatgtcttatttaattaccgtgagtgacatagcctcatgttctgtgggggtcatcagggagggttaggaaaaccacaaacggagcccctgaaagcctcacgtatttcacagagcacgcctgccatcttctccccgaggctgccccaggccggagcccagatacgggggctgtgactctgggcagggacccggggtctcctggaccttgacagagcagctaactccgagagcagtgggcaggtggccgcccctgaggcttcacgccgggagaagccaccttcccaccccttcataccgcctcgtgccagcagcctcgcacaggccctagctttacgctcatcacctaaacttgtactttatttttctgatagaaatggtttcctctggatcgttttatgcggttcttacagcacatcacctctttgcccccgacggctgtgacgcagccggagggaggcactagtcaccgacagcggccttgaagacagagcaaagcgcccacccaggtcccccgactgcctgtctccatgaggtactggtcccttccttttgttaacgtgatgtgccactatattttacacgtatctcttggtatgcatcttttatagacgctcttttctaagtggcgtgtgcatagcgtcctgccctgccccctcgggggcctgtggtggctccccctctgcttctcggggtccagtgcattttgtttctgtatatgattctctgtggttttttttgaatccaaatctgtcctctgtagtattttttaaataaatcagtgtttacattagaa

SEQ ID NO: 13 - protein, 2006 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKNDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 14 - DNA, 8609 bp, Homo sapiens
ggggggagggtggcggctcgatgggggagccgcctccagggggcccccccgccctgtgcccacggcgcggcccctttaagaggcccgcctggctccgtcatccgcgccgcggccacctccccccggccctccccttcctgcggcgcagagtgcgggccgggcgggagtgcggcgagagccggctggctgagcttagcgtccgaggaggcggcggcggcggcggcggcacggcggcggcggggctgtggggcggtgcggaagcgagaggcgaggagcgcgcgggccgtggccagagtctggcggcggcctggcggagcggagagcagcgcccgcgcctcgccgtgcggaggagccccgcacacaatagcggcgcgcgcagcccgcgcccttccccccggcgcgccccgccccgcgcgccgagcgccccgctccgcctcacctgccaccagggagtgggcgggcattgttcgccgccgccgccgccgcgcgggccatgggggccgcccggcgcccggggccgggctggcgaggcgccgcgccgccgctgagacgggccccgcgcgcagcccggcggcgcaggtaaggccggccgcgccatggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagttctatgatgggctcttcccccgcgtgcagcagtggagccaccagcagcgggtgggcgacctcttccagaagctggccagccagctgggtgtgtaccgggccttcgtggacaactacggagttgccatggaaatggctgagaagtgctgtcaggccaatgctcagtttgcagaaatctccgagaacctgagagccagaagcaacaaagatgccaaggatccaacgaccaagaactctctggaaactctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagagcaccggcagctgctgaaggacagcttcatggtggagctggtggagggggcccgcaagctgcgccacgtcttcctgttcaccgacctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagaatgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtagcagcagtcaggggtcaggtgtcaggcccgtcggagctgcctgcagcacatgcgggctcgcccatacccgtgacagtggctgacaagggactagtgagtcagcaccttggcccaggagctctgcgccaggcagagctgagggccctgtggagtccagctctactacctacgtttgcaccgcctgccctcccgcaccttcctcctccccgctccgtctctgtcctcgaattttatctgtggagttcctgctccgtggactgcagtcggcatgccaggacccgccagccccgctcccacctagtgccccagactgagctctccaggccaggtgggaacggctgatgtggactgtctttttcatttttttctctctggagcccctcctcccccggctgggcctccttcttccacttctccaagaatggaagcctgaactgaggccttgtgtgtcaggccctctgcctgcactccctggccttgcccgtcgtgtgctgaagacatgtttcaagaaccgcatttcgggaagggcatgcacgggcatgcacacggctggtcactctgccctctgctgctgcccggggtggggtgcactcgccatttcctcacgtgcaggacagctcttgatttgggtggaaaacagggtgctaaagccaaccagcctttgggtcctgggcaggtgggagctgaaaaggatcgaggcatggggcatgtcctttccatctgtccacatccccagagcccagctcttgctctcttgtgacgtgcactgtgaatcctggcaagaaagcttgagtctcaagggtggcaggtcactgtcactgccgacatccctcccccagcagaatggaggcaggggacaagggaggcagtggctagtggggtgaacagctggtgccaaatagccccagactgggcccaggcaggtctgcaagggcccagagtgaaccgtcctttcacacatctgggtgccctgaaagggcccttcccctcccccactcctctaagacaaagtagattcttacaaggccctttcctttggaacaagacagccttcacttttctgagttcttgaagcatttcaaagccctgcctctgtgtagccgccctgagagagaatagagctgccactgggcacctgcgcacaggtgggaggaaagggcctggccagtcctggtcctggctgcactcttgaactgggcgaatgtcttatttaattaccgtgagtgacatagcctcatgttctgtgggggtcatcagggagggttaggaaaaccacaaacggagcccctgaaagcctcacgtatttcacagagcacgcctgccatcttctccccgaggctgccccaggccggagcccagatacgggggctgtgactctgggcagggacccggggtctcctggaccttgacagagcagctaactccgagagcagtgggcaggtggccgcccctgaggcttcacgccgggagaagccaccttcccaccccttcataccgcctcgtgccagcagcctcgcacaggccctagctttacgctcatcacctaaacttgtactttatttttctgatagaaatggtttcctctggatcgttttatgcggttcttacagcacatcacctctttgcccccgacggctgtgacgcagccggagggaggcactagtcaccgacagcggccttgaagacagagcaaagcgcccacccaggtcccccgactgcctgtctccatgaggtactggtcccttccttttgttaacgtgatgtgccactatattttacacgtatctcttggtatgcatcttttatagacgctcttttctaagtggcgtgtgcatagcgtcctgccctgccccctcgggggcctgtggtggctccccctctgcttctcggggtccagtgcattttgtttctgtatatgattctctgtggttttttttgaatccaaatctgtcctctgtagtattttttaaataaatcagtgtttacattagaa

(19) United States
(12) Patent Application Publication
(10) Pub. No.: US 2014/0295431 A1
(43) Pub. Date: Oct. 2, 2014

(54) METHOD OF ALLELE-SPECIFIC AMPLIFICATION

(71) Applicant: CLARIENT DIAGNOSTIC SERVICES, INC., ALISO VIEJO, CA (US)

(72) Inventor: Paul Christopher Choppa, Aliso Viejo, CA (US)

(73) Assignee: CLARIENT DIAGNOSTIC SERVICES, INC., ALISO VIEJO, CA (US)

(21) Appl. No.: 14/358,120

(22) PCT Filed: Nov. 16, 2012
(86) PCT No.: PCT/US12/65430

§ 371 Date: May 14, 2014

Related U.S. Application Data
(60) Provisional application No. 61/560,866, filed on Nov. 17, 2011.

(30) Foreign Application Priority Data
Dec. 20, 2011 (GB) 1121869.0

(51) Int. Cl. C12Q 1/68

(52) U.S. Cl. CPC C12Q 1/686
USPC 435/6.11

(57) ABSTRACT

A method of selectively producing and amplifying a cDNA sequence of a target allele of a gene, wherein the target allele is a mutant allele or is a specific allele of a polymorphic gene, the method comprising: (a) providing a sample comprising an mRNA transcript of the target allele; (b) performing a reverse-transcription reaction to generate a cDNA sequence from the mRNA transcript, and (c) amplifying the cDNA of the target allele; wherein the reverse-transcription reaction is selective for reverse transcription of the mRNA transcript of the target allele over an mRNA transcript of an alternative allele of the same gene.

### FIELD OF THE INVENTION

The present invention relates to a method of selectively producing and amplifying a cDNA sequence of a target allele of a gene. The present invention also relates to a kit for selectively producing and amplifying a cDNA sequence of a target allele of a gene.

### BACKGROUND OF THE INVENTION

Risk of disease and response to treatment varies from person to person. This is to due to variation in human genetic coding, interactions between genes and the environment over a lifetime, and the unique signature of the immune system. Defining the scope and nature of human biological variation has allowed, and will continue to allow, assessments to be made regarding disease diagnosis, disease prognosis and the targeting of medical treatments to those that will most likely benefit.

Variations in DNA between individuals can be caused by DNA mutations. Mutagenesis can lead to sudden and spontaneous changes in a cell and can arise from a number of different possible causes, including radiation, viruses, transposons and mutagenic chemicals, as well as errors that occur during cell division or DNA replication. They can also be induced by the organism itself by cellular processes such as hypermutation. Mutations exist in different forms, such as point mutations, insertions and deletions. Mutations such as point mutations that occur within protein coding regions of gene can lead to erroneous codon codes which can e.g. code for a different amino acid or code for a stop codon resulting in a truncated form of a protein. Mutations may also lead to frameshifts caused by the insertion or deletion of nucleotides, resulting in a completely different translation from the wild-type sequence. Mutations may lead to loss of function of a protein, gain of function, or may act antagonistically to the wild-type allele.

Mutations can provide important genetic markers for disease diagnosis or prognosis. Furthermore, the identification of mutations can play a significant role in helping to tailor drugs and drug regimens to particular genotypes.

Mutational profiling of key cancer pathway genes is becoming common practice in the way therapies are being selected for patient care. Some alterations have been shown to increase sensitivity to a certain drug while other mutations result in decreased sensitivity or even resistance to a given therapy. There are a number of reports in the literature that document the increased sensitivity to erlotinib seen in NSCLC patients with to an L858R and other EGFR mutations. Additionally, resistance to imatinib seen in patients with CML has been associated with mutations in the kinase domain of the c-abl gene involved in the BCR-ABL fusion gene. Although identification of these mutations has become important for patient care, these therapies were not originally developed to target the specific alterations. In more recent years specific alterations identified in subsets of patients are being used to develop targeted therapies against the key alterations. Two recent examples of the trend toward such targeted therapies are crizotinib and vemurafenib for patients with EML4-ALK fusions and BRAF V600E mutations, respectively. Vemurafenib has been approved by the Food and Drug Administration to treat patients with metastatic melanoma who have a BRAF V600E mutation. The BRAF protein is normally involved in cell growth regulation, but is mutated in about half of patients with late-stage melanomas. Vemurafenib is a BRAF inhibitor that is able to block the function of the V600E-mutated BRAF protein from driving the proliferation of cancer cells. As more therapies are developed against specific alterations the increased need for sensitive and specific mutational profiling methodologies is becoming more important.

Nucleic acid polymorphisms can also contribute to the genetic diversity between individuals. Polymorphisms can take several forms, including single nucleotide substitutions, nucleotide insertions, and nucleotide deletions. In the case of insertions and deletions, the insertion or deletion of one or more nucleotides at a position in a gene may be present.

Single nucleotide polymorphisms (SNPs) represent an abundant form of genetic variation in humans. SNP patterns are likely to influence many human phenotypes. Consequently, large scale association studies based on SNP genotyping are expected to help identify genes affecting complex diseases and responses to drugs or environmental chemicals. SNPs can provide important genetic markers for disease diagnosis or prognosis. Furthermore, the identification of SNPs (and other genetic polymorphisms) can play a significant role in helping to tailor drugs and drug regimens to particular genotypes.

As a consequence of the clear impact that pharmacogentics can, and will, have on the healthcare industry, there is a pressing need to develop improved methods of genotype testing.

Various methods of allele discrimination methods are known in the art. Examples of such methods include allele specific hybridisation, allele-specific single-base primer extension, allele specific enzymatic cleavage, and allele-specific polymerase chain reaction (AS-PCR).

Allele specific hybridisation discriminates between alleles at a SNP locus using allele-specific oligonucleotide probes. Stringency conditions are employed such that a single-base mismatch is sufficient to prevent hybridisation of the non-matching probe. In allele-specific single-base extension, primers are designed that anneal one nucleotide upstream of the polymorphic or mutant site. In this method, allele discrimination depends on the ability of this perfectly-annealed primer to be extended. Allele specific enzymatic cleavage employs fragment length polymorphism (RFLP) analysis. An RFLP is generated when a mutation/SNP occurs at a restriction endonuclease recognition sequence, and one allele preserves the sequence while the other destroys it. The presence of a mutation/SNP can be detected from the number of cleavage products after application of a restriction enzyme.

Allele-specific polymerase chain reaction is a powerful method in which allele discrimination is achieved by allele-specific primer annealing, followed by PCR amplification. Allele specific PCR is typically performed on DNA samples using primers that have a complimentary nucleotide in the primer (e.g. in the 3′ position) in order to selectively amplify the intended target. Although this methodology works well, it often requires a fair amount of optimisation and knowledge about primer/template interactions in order to obtain the required specificity. The issue many times is that the discriminatory power of the single nucleotide change may not be sufficient to inhibit amplification of the wild-type allele to some degree. There are a number of ways to manipulate the reaction and reagents reported in the literature in order to attempt to increase specificity. However, there are a number of different factors to consider other than just the affinity of the primer for the intended template. One such factor is the amount of mutant target present in the background of wild-type alleles. Wild-type alleles that are present in the reaction mix make it much more difficult to selectively amplify the intended mutant target as the wild-type alleles tend to bind primers and to some degree generate signals that are indistinguishable from the intended amplification.

Renaud et al. (Journal of Clinical Virology; 49 (2010); 21-25) describe a diagnostic assay employing an allele-specific reverse transcriptase-PCR (AS-RTPCR) assay that targets to the H275Y oseltamivir resistant mutation in 2009 pandemic influenza A.H1N1 virus. The method employed by Renaud uses a two-step RT-PCR reaction employing a common reverse primer and probe and two-allele-specific forward primers (wild-type and mutant) which are designed to include the SNP of interest at the 3′ end. However, since the reverse primer (the primer for the reverse transcription step) was common for the two alleles, the reverse transcription reactions were not discriminatory for the transcripts of the mutant versus wild-type. Accordingly, any discriminating ability of the method required discrimination at the cDNA level.

Against this background, there is pressing need to develop improved methods of genotype testing, for example to distinguish between mutant and wild-type alleles. In particular, new methods are required that improve the sensitivity and specificity to enable accurate and efficient routine testing procedures.

### SUMMARY OF THE INVENTION

The present invention relates to a method of selectively producing and amplifying a target allele of a gene.

More particularly, the present invention provides a method of selectively producing and amplifying a cDNA sequence of a target allele of a gene, the method comprising:
a) providing a sample comprising an mRNA transcript of the target allele;
b) performing a reverse-transcription reaction to generate a cDNA sequence from the mRNA transcript, and
c) amplifying a cDNA sequence of the target allele generated in step (b);
   wherein the reverse-transcription reaction in step (b) is selective for reverse transcription of the mRNA transcript of the target allele over an mRNA transcript of an alternative allele of the same gene.

The target allele may be a mutant allele or a specific allele of a polymorphic gene. Thus, the present invention provides a method of selectively producing and amplifying a cDNA sequence of a target allele of a gene, wherein the target allele is a mutant allele or is a specific allele of a polymorphic gene, the method comprising:
a) providing a sample comprising an mRNA transcript of the target allele;
b) performing a reverse-transcription reaction to generate a cDNA sequence from the mRNA transcript, and
c) amplifying a cDNA sequence of the target allele generated in step (b);
   wherein the reverse-transcription reaction in step (b) is selective for reverse transcription of the mRNA transcript of the target allele over an mRNA transcript of an alternative allele of the same gene.

The present invention thus relates to a method that helps to eliminate the presence of unwanted alleles (e.g. wild-type alleles) in the nucleic acid (e.g. cDNA) population. This is accomplished by selectively turning the allele target (e.g. mutant allele) mRNA into potential targets for amplification, while leaving the unwanted allele (e.g. wild-type allele) in the form of RNA which is not a substrate for amplification. This conversion of (e.g. mutant) allele mRNA to cDNA happens in the initial reverse transcription step of the reaction. As the reaction proceeds to amplification by, for example a polymerase chain reaction (PCR), the only targets present in the sample are the target (e.g. mutated) copies of cDNA which helps to eliminate the potential for mis-priming.

The present invention thus allows for significant advantages over conventional allele specific PCR. Firstly, by only priming the transcript of interest (e.g. mutant), the only cDNA that is generated is from the transcripts of interest (e.g. mutant transcripts), in effect eliminating the potential for mis-priming and reducing unwanted background signals. Secondly, the method increases the number of targets per cell equivalence over the single copy of DNA that would be available using standard AS-PCR e.g. if the gene is transcribed at a rate of more than one copy from the allele of interest (e.g. mutated allele) per cell. This is particularly advantageous as the number of cells available for testing becomes limited, as is often the case with e.g. circulating tumour cell (CTC) analysis and other small biopsy samples. The present inventors have demonstrated this successfully using patient samples comprising the V600E mutation. When performing a traditional allele specific PCR, the reaction can generate a significant amount of background in known wild-type samples. Additionally, the level of sensitivity is in the region of 1-5%. However, when using the method of the present invention, the background signal is improved and a significant gain in sensitivity is achieved. Thus, the method of the present invention can serve as a way to increase available targets for detection of mutations in rare event populations as well as increasing the sensitivity and specificity of routine allele-specific testing.

In addition to helping in the selection of therapies and the determination of diagnosis or prognosis based on a mutation or polymorph (e.g. SNP) profile, the present invention may also be clinically significant in being able to determine the presence of an active gene carrying a mutation or polymorph by detecting the mutation or polymorph at the transcript level. This provides an additional level of confidence that the mutated allele is actually driven from an active promoter and therefore produces the targeted protein. Accordingly, the invention as described herein may also serve as an attractive alternative in the absence of robust antibodies to detect variant proteins.

Additionally, the invention described herein may provide valuable information about the quantity of a mutant transcript present in a sample as a means to monitor drug efficacy and disease progression. Quantitative DNA based PCR for mutations associated with hematologic malignancies such as abl T315I and JAK2 V617F are currently in use today. Although there is currently no targeted therapy for patients with a JAK2 V617F mutation, there are a number of development efforts underway which target this mutation in patients with Polycythemia Vera. Monitoring the efficacy of a JAK2 inhibitor with a quantitative JAK2 V617F determination at the transcript level may provide clinically relevant information similar to how BCR-ABL RT-PCR is used for monitoring therapy and disease progression in patients with CML today.

In a preferred embodiment of the present invention, the target allele is a mutant allele. In a further preferred embodiment, the target allele is a mutant allele and the alternative allele is the wild-type allele. The mutant allele may be, for example, the result of a point mutation, nucleotide insertion(s) or a nucleotide deletion(s). In a preferred embodiment, the method is used to amplify a mutant allele comprising a specific point mutation that is not present in the alternative allele. In a preferred embodiment, the mutant allele comprises a single nucleotide substitution that is not present in the alternative allele i.e. the method can discriminate between a sequence containing the nucleotide substitution versus the corresponding sequence that does not contain the nucleotide substitution.

The target allele may also be a specific allele of a polymorphic gene comprising a polymorphic site, the target allele and alternative allele differing in base composition at the polymorphic site. The polymorphism may be, for example, in the form of a single nucleotide polymorphism (SNP), nucleotide insertion(s) or nucleotide deletion(s). In a preferred embodiment, the polymorphic site is a SNP site.

In a preferred embodiment, the reverse-transcription reaction comprises (i) annealing a reverse primer to a region of the mRNA transcript of the target allele comprising a target site and (ii) extending the reverse primer to generate a cDNA sequence from the mRNA transcript of the target allele, wherein the mRNA transcript of the target allele and the mRNA of the alternative allele differ in base composition at the position of the target site, and wherein selectivity for reverse transcription of the target allele mRNA over the alternative allele mRNA is achieved by the presence of one or more bases in the reverse primer which are complementary to the mRNA sequence at the target site of the target allele but which establish a mis-match at the position of the target site in the alternative allele. The target site may be, for example, a mutation site or a polymorphic (e.g. SNP) site, as described herein.

The reverse primer used in the present invention may bind the target site with full complementarity to the mRNA of the target allele or with one or more base mis-matches may be present. In a particularly preferred embodiment, the reverse primer binds with full complementarity (i.e. no base-base mis-matches) to the mRNA of the target allele.

The selectivity for reverse transcription of the target allele mRNA over the alternative allele mRNA can be achieved, at least in part, by a base at the 3′ end of the reverse primer which establishes a mis-match with the mRNA sequence of the alternative allele but which base-pairs with the mRNA sequence of the target allele.

In a particularly preferred embodiment, the amplification step comprises performing a polymerase chain reaction (PCR) on the generated cDNA sequence of the target allele. In a preferred aspect of this embodiment, the reverse transcription reaction and PCR reaction employ the same reverse primer. The forward primer and reverse primer employed in the PCR reaction may each bind to a region of the target allele derived from the same exon and/or the reverse transcription reaction and PCR reaction are carried out using the same enzyme, optionally wherein the enzyme is rTth.

In an embodiment of the present invention, the target allele that is selectively amplified by the present invention may be an allele of HER2, PI3K (PIK3CA), KRAS, EGFR, c-MET, MEK (MEK1 or MEK2), PTEN, NRAS, HRAS, FGFR1, JAK2, ABL (also known as ABL1 and c-able oncogene 1, non-receptor tyrosine kinase), BRAF or ALK. For example, the target allele may be selected from the group consisting of BRAF V600E, BRAF V600D, BRAF V600R, BRAF V600K, EGFR L858R, EGFR T790M, ALK C1156Y and ABL T315I. In one embodiment, the targets recited herein may be part of gene fusion constructs encoding fusion proteins (e.g. EML4-ALK fusions and BCR-ABL fusions).

In a particularly preferred embodiment of the present invention, the selectively amplified cDNA is detected and/or quantified e.g. by real-time PCR.

In a further aspect of the invention, the presence of the target allele is predictive of a diagnosis and/or a prognosis of a subject from which the sample is taken. In an embodiment of this aspect, the method may comprise detecting and/or quantifying the amplified cDNA of the target allele and assessing from the detection/quantitation of the amplified cDNA a diagnosis and/or a prognosis of the subject.

In a further aspect, the sample that is analysed in the present invention is from a subject known to have, or suspected to have, a disease, and the presence of the target allele is predictive of how the subject will respond to administration of a drug to treat the disease. In an embodiment of this aspect, the method may comprise detecting and/or quantitating the amplified cDNA of the target allele and assessing from the detection/quantitation of the amplified cDNA the likelihood of success of treating the subject with the drug. For example, the disease may be cancer, the target allele can be the mutant allele of the human BRAF gene encoding the V600E mutation and the drug can be vemurafenib.

The present invention also provides a kit for selectively producing and amplifying a cDNA sequence of a target allele of a gene by reverse transcription PCR, wherein the target allele is a mutant allele or is a specific allele of a polymorphic gene, and wherein the kit comprises: (i) a reverse primer specific to a region of an mRNA transcript of the target allele comprising a target site, wherein the mRNA transcript of the target allele of the gene and the mRNA of an alternative allele of the gene differ in base composition at the position of the target site, and wherein the reverse primer comprises one or more bases which are complementary to the mRNA sequence at the target site of the target allele but which establish a mis-match at the position of the target site in the alternative allele; (ii) a forward primer specific for an upstream region of the target allele; (iii) a reverse transcriptase; and (iv) a DNA polymerase.

In a preferred embodiment, the reverse transcriptase and the DNA polymerase of the kit are the same enzyme.

In a further aspect, provided herein is a method of detecting for the presence of a gene mutation, the method comprising:
a) providing a sample comprising an mRNA transcript;
b) contacting the sample with reagents capable of performing a reverse-transcription reaction when mRNA containing the mutation is present, thereby generating a cDNA sequence from the mRNA transcript when mRNA containing the mutation is present; and
c) amplifying, if present, any of the said cDNA sequence generated in step (b);
   wherein the reverse-transcription reaction in step (b) is selective for reverse transcription when the mRNA transcript containing the mutation is present over the alternative transcript of the gene that does not contain the mutation.

The reagents in step (b) of this aspect will typically comprise a reverse primer which is selective for reverse transcription of the mRNA containing the mutation by the presence of a base in the reverse primer which is complementary to the mRNA base containing the mutation but which establishes a mismatch in the alternative transcript. The base is preferably at the 3′ end of the reverse primer. Step (c) preferably comprises annealing a forward primer to the cDNA sequence and performing a polymerase chain reaction (PCR) on the cDNA sequence. The reverse transcription reaction and PCR reaction may employ the same reverse primer. The mutation may be any of the mutations described above, and may be for example mutations in HER2, PI3K (PIK3CA), KRAS, EGFR, c-MET, MEK, PTEN, NRAS, HRAS, FGFR1, JAK2, ABL, BRAF or ALK.

In a further aspect, the present invention provides a method of detecting for the absence of a gene mutation, the method comprising:
a) providing a sample comprising an mRNA transcript from the gene;
b) performing a reverse-transcription reaction to generate a cDNA sequence from the mRNA transcript; and
c) amplifying the cDNA sequence generated in step (b);
   wherein the reverse-transcription reaction in step (b) is selective for reverse transcription of the mRNA transcript when the mutation is absent over the corresponding mRNA transcript containing the mutation.

In this aspect, the reverse transcription reaction may comprise:
(i) annealing a reverse primer to a region of the mRNA containing the mutation site; and
(ii) extending the reverse primer to generate a cDNA sequence from the mRNA transcript; wherein selectivity for reverse transcription of the mRNA is achieved, at least in part, by the presence of a base which establishes a mismatch in the mRNA transcript having the mutation at the mutation site. The base is preferably at the 3′ end of the reverse primer. Step (c) preferably comprises annealing a forward primer to the cDNA sequence and performing a polymerase chain reaction (PCR) on the cDNA sequence. The reverse transcription reaction and PCR reaction may employ the same reverse primer. The mutation may be any of the mutations described above, and may be for example mutations in HER2, PI3K (PIK3CA), KRAS, EGFR, c-MET, MEK, PTEN, NRAS, HRAS, FGFR1, JAK2, ABL, BRAF or ALK.

### BRIEF DESCRIPTION OF THE DRAWINGS

FIG. 1a and FIG. 1b show amplification plots generated from sample 1 using DNA (FIG. 1a) and mRNA (FIG. 1b) as the starting material. FIG. 1a has a Ct value of 31.8 and FIG. 1b has a Ct value of 25.8.

FIGS. 2a and 2b show amplification plots generated from sample 2 using DNA (FIG. 2a) and mRNA (FIG. 2b) as the starting material. FIG. 2a has a Ct value of 24.4 and FIG. 2b has a Ct value of 23.6.

FIGS. 3a and 3b show amplification plots generated from sample 3 using DNA (FIG. 3a) and mRNA (FIG. 3b) as the starting material. FIG. 3a has a Ct value of 33.2 and FIG. 3b has a Ct value of 25.7.

FIGS. 4a and 4b show amplification plots generated from sample 4 using DNA (FIG. 4a) and mRNA (FIG. 4b) as the starting material. FIG. 4a has a Ct value of 25.4 and FIG. 4b has a Ct value of 25.3.

FIGS. 5a and 5b show amplification plots generated from sample 5 using DNA (FIG. 5a) and mRNA (FIG. 5b) as the starting material. FIG. 5a has a Ct value of 34 and FIG. 5b has a Ct value of 26.3.

### DETAILED DESCRIPTION OF THE INVENTION

The following definitions apply to the present invention:

The term “allele” refers to a particular form of a genetic locus, distinguished from other forms by its particular nucleotide or amino acid sequence.

The term “target allele” refers to an allele that is to be selectively amplified using the method of the present invention. The target allele may represent a mutant or polymorphic variant that is present in a population at lower frequency. The target may also be a wild-type allele. In some cases, two or more alleles of a given gene may have the same mutation or polymorphism in common that is to be detected by the method of the present invention. In such cases, a target allele may comprise two or more alleles that share a mutation of interest.

The term “alternative allele” refers to an alternative allele of the target allele gene. The alternative allele will code for an mRNA sequence that differs from the mRNA sequence coded by the target allele. The method of the present invention is capable of selectively producing and amplifying cDNA of the target allele over producing and amplifying cDNA the alternative allele when the target allele and alternative allele are in the same sample. The mRNA transcript sequence from the target allele and the mRNA transcript sequence from the alternative allele may, for example, differ only in the base or bases present (or absent) at a single mutant site or polymorphic site.

The term “gene” refers to a hereditary unit consisting of a sequence of DNA that occupies a specific location on a chromosome and determines a particular characteristic in an organism.

The term “locus” refers to a location on a chromosome or DNA molecule corresponding to a gene or a physical or phenotypic feature.

The term “nucleic acid” refers to a single stranded or double stranded DNA or RNA molecule including natural nucleic acids found in nature and/or modified, artificial nucleic acids having modified backbones or bases, as are known in the art.

The term “polymorphic site” refers to a position within a locus at which at least two alternative bases or sequences are found in a population.

The term “polymorphism” refers to the sequence variation observed in an individual at a polymorphic site. Polymorphisms include nucleotide substitutions, insertions, deletions, and may, but need not, result in detectable differences in gene expression or protein function.

The term “primer” refers to a molecule that physically hybridizes with a target nucleic acid. The primer is capable of being extended in an amplification reaction such as a PCR reaction or in a reverse-transcription reaction. Typically, a primer can be made from, or comprise of, any combination of nucleotides or nucleotide derivatives or analogs available in the art. More typically, a primer will be in the form of an oligonucleotide. Primers may also contain one or more nucleotide alternatives or modified bases to add increased specificity and/or disrupt the efficiency of primer extension in the presence of a mis-match. Alternative bases used to enhance specificity may include Locked Nucleic Acid (LNA) bases, Peptide Nucleic Acid (PNA) bases and Inosine. The primer may be unlabelled or labelled with a detection marker.

The present invention provides a method of selectively producing and amplifying a cDNA sequence of a target allele of a gene, wherein the target allele is a mutant allele or is a specific allele of a polymorphic gene, the method comprising:
a) providing a sample comprising an mRNA transcript of the target allele;
b) performing a reverse-transcription reaction to generate a cDNA sequence from the mRNA transcript, and
c) amplifying a cDNA sequence of the target allele generated in step (b);
   wherein the reverse-transcription reaction in step (b) is selective for reverse transcription of the mRNA transcript of the target allele over an mRNA transcript of an alternative allele of the same gene. Accordingly, when the target allele of the gene and the alternative allele of the gene are present in the same sample, the method of the present invention is able to selectively produce and amplify a cDNA sequence of a target allele of a gene over producing and amplifying a cDNA sequence of the alternative allele. In one embodiment, the target allele and the alternative allele DNA and/or RNA are both present in the sample. However, it will be appreciated in some cases the method of the present invention can be carried out on a sample where it is not known whether both the target allele and alternative allele nucleic acid are present in the sample e.g. the sample may contain target allele but not the alternative allele (or vice-versa). The method of the present invention is also advantageous in such cases since detection of amplification product resulting from the method of the present invention can be used to confirm the presence of the target allele.

For example, the method of the present invention may be used to detect for the presence of a gene mutation, the method comprising:
a) providing a sample comprising an mRNA transcript;
b) contacting the sample with reagents capable of performing a reverse-transcription reaction when mRNA containing the mutation is present, thereby generating a cDNA sequence from the mRNA transcript when mRNA containing the mutation is present; and
c) amplifying, if present, any of the said cDNA sequence generated in step (b);
   wherein the reverse-transcription reaction in step (b) is selective for reverse transcription when the mRNA transcript containing the mutation is present over the alternative transcript of the gene that does not contain the mutation.

The method may also be used to detect for the absence of a gene mutation, the method comprising:
a) providing a sample comprising an mRNA transcript from the gene;
b) performing a reverse-transcription reaction to generate a cDNA sequence from the mRNA transcript; and
c) amplifying the cDNA sequence generated in step (b);
   wherein the reverse-transcription reaction in step (b) is selective for reverse transcription of the mRNA transcript when the mutation is absent over the corresponding mRNA transcript containing the mutation.

In the reverse transcription reaction, a DNA molecule (termed a complementary DNA molecule, which can be abbreviated to a “cDNA molecule”) is generated from a single stranded RNA template through the enzyme reverse transcriptase. Generating cDNA from mRNA is well known in the art. The cDNA sequence may be generated from the full length of the mRNA sequence or a portion of the mRNA sequence. A skilled person can readily determine the appropriate annealing and extension temperatures from the primer sequence, mRNA template and choice of reverse transcriptase using procedures well known in the art. In one embodiment, reverse transcription is executed by rTth.

The selectivity of the method can be achieved, for example, by annealing a reverse primer to a region of the mRNA transcript of the target allele that contains the only difference in sequence between the mRNA transcript of the target allele and the mRNA transcript of the alternative allele.

In a preferred embodiment, the selectivity of the method can be achieved by annealing a reverse primer to a region of the mRNA transcript of the target allele comprising a target site and extending the reverse primer to generate a cDNA sequence from the mRNA transcript of the target allele, wherein the mRNA transcript of the target allele and the mRNA of the alternative allele differ in base composition at the position of the target site. In this way, the selectivity for reverse transcription of the target allele mRNA over the alternative allele mRNA is achieved by the presence of one or more bases in the reverse primer which are complementary to the mRNA sequence at the target site when the primer is hybridized to the mRNA transcript of the target allele but which establishes a mismatch at the position of the target site in the alternative allele. A mismatch may be established, for example, by the disruption or removal of one or more non-covalent bonds, such as one or more hydrogen bonds e.g. by disrupting or removing a Watson-Crick base pair.

A person skilled in the art would be able to generate allele-specific primers using methods known in the art. In a preferred embodiment, and as exemplified in the specific examples disclosed herein, the reverse primer is designed to have a residue at the 3′ terminus of the primer that is complimentary to the mRNA of the target allele and not to mRNA of another (or the other) alternative allele of the gene. Thus, the reverse primer may be designed to have a base at the 3′ terminus of the primer that, when the primer is annealed to the mRNA of the target allele, is complementary to a base present at the target site (e.g. mutant site or polymorphic site) of the target allele but is not complementary to a base present in the corresponding position of the alternative allele. Modifications to the primer adjacent to the 3′ terminal base may also create enough disruption of the primer binding efficiency to effectively disable primer extension. Additionally, modified bases known to increase hybridization stringency such as LNA and PNA bases may also be substituted at strategic positions of the primer. The presence of the “mismatch” (e.g. by removing or disrupting the formation of a base-pair) at the 3′ end of the reverse primer disrupts the ability of the reverse transcriptase to extend the primer. A skilled person would readily be able to confirm whether a reverse primer has the desired selectivity by performing a reverse transcription reaction in the presence of target allele and alternative allele and detecting the level of cDNA production.

In a preferred embodiment, the alternative allele is complementary along the full length of the reverse primer with the exception of a single non-complementary mis-match between the base at the 3′ end of the reverse primer and the corresponding base of the alternative allele. However, the alternative allele may be complementary along the full sequence of the reverse primer with the exception of two or more base-pair mis-matches (e.g. 2, 3, 4, 5 or more) between the reverse primer and the corresponding base of the alternative allele.

In a preferred embodiment of the present invention, the reverse primer hybridizes to an mRNA transcript of the target allele with full complementarity to the mRNA of the target allele. By this is meant that each base of the reverse primer forms a base-pair with a complementary base on mRNA transcript when the primer is hybridized to the mRNA transcript.

The reverse primer of the present invention can be a nucleic acid sequence, preferably a DNA oligonucleotide. The primer is of sufficient length to enable reverse transcription of an mRNA transcript of the target allele. The primer may be, for example, in the range of 10-50 nucleotides in length, preferably about 10-35 nucleotides, more preferably about 10-30 nucleotides in length.

In the method of the present invention, the cDNA molecule that is generated from the mRNA transcript of the target allele is subjected to an amplification reaction. It should be noted that references throughout this disclosure to amplifying a cDNA sequence of the target allele generated in step (b) encompasses amplification of either the complete cDNA sequence generated in step (b) or a part of the cDNA sequence generated in step (b). Amplification of DNA is an established procedure in molecular biology and can be carried out by many alternative methods known in the art. Example amplification methods include thermal cycler based amplification with thermostable enzymes (e.g. polymerase chain reaction (PCR), ligase chain reaction (LCR), in-situ amplification, long distance PCR, digital PCR, real-time PCR, multiplex PCR and ligation-dependent probe amplification), and isothermal amplification (e.g. strand displacement amplification (SDA), real-time strand displacement amplification, loop mediated isothermal amplification, ligation mediated rolling circle amplification, rolling circle amplification, and multiple displacement amplification).

Descriptions of amplification techniques can be found in, among other places, Sambrook and Russell; Sambrook et al.; Ausbel et al.; PCR Primer: A Laboratory Manual, Diffenbach, Ed., Cold Spring Harbor Press (1995); The Electronic Protocol Book, Chang Bioscience (2002) (“The Electronic Protocol Book”); Msuih et al., J. Clin. Micro. 34:501-07 (1996); The Nucleic Acid Protocols Handbook, R. Rapley, ed., Humana Press, Totowa, N.J. (2002)(“Rapley”); U.S. Pat. No. 6,027,998; Barany et al., PCT Publication No. WO 97/31256; Wenz et al., PCT Publication No. WO 01/92579; Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticid proc/ussymp6proc/blegrad.html); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); and Landegren et al., Science 241:1077-80 (1988).

In a particularly preferred embodiment, the amplification method employed in the present invention is a PCR-based amplification method. Polymerase chain reaction (PCR) is very widely known in the art. For example, U.S. Pat. Nos. 4,683,195, 4,683,202, and 4,800,159; K. Mullis, Cold Spring Harbor Symp. Quant. Biol., 51:263-273 (1986); and C. R. Newton & A. Graham, Introduction to Biotechniques: PCR, 2.sup.nd Ed., Springer-Verlag (New York: 1997), the disclosures of which are incorporated herein by reference, describe processes to amplify a nucleic acid sample target using PCR amplification extension primers which hybridize with the sample target.

Using PCR, the cDNA is amplified exponentially using a polymerase e.g. a DNA polymerase. PCR requires forward and reverse extension primers which hybridize with the sample target. As the PCR amplification primers are extended, using a DNA polymerase (preferably thermostable), more sample target is made so that more primers can be used to repeat the process, thus amplifying the sample target sequence. Typically, the reaction conditions are cycled between those conducive to hybridization and nucleic acid polymerization, and those that result in the denaturation of duplex molecules. To briefly summarize, in the first step of the reaction, the nucleic acid molecules of a sample are transiently heated, in order to denature double stranded molecules. Forward and reverse primers are present in the amplification reaction mixture at an excess concentration relative to the sample target. When the sample is cooled to a temperature conducive to hybridization and polymerization, the primers hybridize to the complementary sequence of the nucleic acid molecule at a position 3′ to the sequence of the region desired to be amplified that is the complement of the sequence whose amplification is desired. Upon hybridization, the 3′ ends of the primers are extended by the polymerase. The extension of the primer results in the synthesis of a DNA molecule having the exact sequence of the complement of the desired nucleic acid sample target. The PCR reaction is capable of exponentially amplifying the desired nucleic acid sequences, with a near doubling of the number of molecules having the desired sequence in each cycle. Thus, by permitting cycles of denaturation, hybridization, and polymerization, an exponential increase in the concentration of the desired nucleic acid molecule can be achieved. A preferred physical means for strand separation involves heating the nucleic acid until it is completely (>99%) denatured. Typical heat denaturation involves temperatures ranging from about 80° C. to about 105° C., for times ranging from a few seconds to minutes.

In the present invention, the template for amplification is the cDNA strand produced during the reverse transcription step. Accordingly, the PCR reaction would typically require a forward primer that anneals to the cDNA strand produced during the reverse transcription step and which is then extended using an enzyme with DNA polymerase activity to produce the complement cDNA strand. The resulting cDNA strand can then be denatured and the forward primer and a reverse primer annealed to the respective cDNA strands to allow further extension. The primers are then extended by the polymerase to replicate the cDNA sequences, and the process is then repeated multiple times.

In a particularly preferred embodiment, the reverse primer used for the reverse transcription step is also used as the reverse primer for the PCR amplification step. This is advantageous in that the reverse transcription step and the PCR amplification can be carried out using a single cocktail of reagents, thereby allowing a “one-step” reaction. The cocktail of reagents may comprise a reverse transcriptase enzyme for the reverse transcription step and a DNA polymerase enzyme for the PCR reaction. However, in a preferred aspect of the invention, a single enzyme is used that is able to perform the enzymatic steps in both the reverse transcription reaction and the PCR reaction. An example of such an enzyme that is configured for DNA polymerization and reverse transcription that can be used in the present invention is rTth.

In a further embodiment, the forward primer and reverse primer are specific for the same exon.

In an alternative embodiment, for example where contaminating DNA is present, primer pairs may be employed that span intron-exon boundaries to further prevent genomic DNA from being amplified. For example, the forward primer may hybridize to a region of the cDNA derived from two different exons. Alternatively, each member of a primer pair may span different exons.

The term “sample” is used in a broad sense herein and is intended to include a wide range of biological materials as well as compositions derived or extracted from such biological materials. Exemplary such materials or samples include whole blood; red blood cells; white blood cells; buffy coat; hair; nails and cuticle material; swabs, including but not limited to buccal swabs, throat swabs, vaginal swabs, urethral swabs, cervical swabs, throat swabs, rectal swabs, lesion swabs, abcess swabs, nasopharyngeal swabs, and the like; urine; sputum; circulating tumour cells (CTCs); exosomes; microsomes; cell free nucleic acid; saliva; semen; lymphatic fluid; amniotic fluid; cerebrospinal fluid; peritoneal effusions; pleural effusions; fluid from cysts; synovial fluid; vitreous humor; aqueous humor; bursa fluid; eye washes; eye aspirates; plasma; serum; pulmonary lavage; lung aspirates; and tissues, including but not limited to, liver, spleen, kidney, lung, intestine, brain, heart, muscle, pancreas, biopsy material, and the like. The skilled artisan will appreciate that lysates, extracts, or material obtained from any of the above exemplary biological samples are also within the scope of the invention. Tissue culture cells, including explanted material, primary cells, secondary cell lines, and the like, as well as lysates, extracts, or materials obtained from any cells, are also within the meaning of the term biological sample as used herein. In one embodiment the sample is derived from a tissue section. Tissue sections may be formalin-fixed paraffin embedded tissue sections. Such sections may be incubated in digestion buffer to release the cellular content.

The samples used in the practice of the present invention may be obtained or derived from any source that contains, or is considered to potentially contain an mRNA transcript of a target allele or a part of said mRNA transcript. The mRNA-containing sample may, for example, be obtained or derived from any mammal. Preferably the mRNA-containing sample is obtained from or derived from a human. In one embodiment, the mRNA is obtained or derived from a subject known to have or suspected of having a disease. An example of such a disease is cancer. The cancer may be prostate, breast, lung, ovarian, pancreatic, bowel, colon, stomach, skin cancer, metastatic melanoma, or a brain tumour or malignancy affecting the bone marrow (including the leukaemias) and lymphoproliferative systems, such as Hodgkin's or non-Hodgkin's lymphoma.

In a preferred embodiment of the present invention, the sample to be used in the method of the present invention can be pre-processed, for example to remove contaminating DNA and/or purify RNA (including mRNA) in the sample. Methods for DNA removal are known in the art, such as acid phenol:chloroform extraction or Lithium chloride precipitation, and can be followed DNase digestion. In one embodiment the DNA and RNAs may be separated using column-based extraction protocols. Standard methodologies for extracting nucleic acid in a test sample are well known in the art (see, for example, Sambrook et al. “Molecular Cloning—A Laboratory manual”, second edition. Cold Spring Harbor, N.Y. (1989)). In a particularly preferred embodiment, the sample contains RNA that has been isolated from contaminating DNA. Isolation of RNA is a routine procedure in the art and there are multiple commercially available kits for this purpose (e.g. Strategene RNA Isolation kit).

The method of the present invention may further comprise detecting the amplified cDNA generated by reverse transcription of an mRNA transcript of the target allele. The method of detection may vary depending on the method used for the amplification step.

Many methods of detecting DNA sequences are known in the art. The simplest method of detection of nucleic acid amplification products is agarose gel electrophoresis. Products are separated based on mass by electrophoresis through an agarose gel. The gels are then stained with ethidium bromide, or an alternative such as SYBR green, which cause nucleic acids to fluoresce under UV light. Stains used in agarose gel electrophoresis give off a fluorescent signal when intercalated into DNA, but not when unincorporated. Positive results are those in which a band of the appropriate size is present, while negative results lack the appropriate band.

A preferred method of detecting the amplified cDNA in the present invention is employing real-time PCR. Real-time PCR allows amplification of the target nucleic acid to be visualized in real time. Real-time detection can be accomplished in a number of alternative ways. A first way is through the use of a DNA intercalating fluorescent dye. This type of reaction uses two primers just like a standard PCR, but also requires the addition of an intercalating dye. An example of such a dye is SYBR green. In this type of assay, as the specific target amplifies, more dye becomes incorporated into DNA and the fluorescent signal increases. A second type of real-time PCR detection also uses two primers, but employs a fluorescently labeled oligonucleotide probe. This second method works on the principle that a fluorescent dye (the reporter) is attached to one end of the oligonucleotide and a quencher, which absorbs light emitted from the reporter when in close proximity to it, is bound to the other end. The close proximity of the reporter to the quencher prevents detection of its fluorescence; breakdown of the probe by the 5′ to 3′ exonuclease activity of the amplification polymerase breaks the reporter-quencher proximity and thus allows unquenched emission of fluorescence, which can be detected after excitation with a light source. An increase in the product targeted by the reporter probe at each PCR cycle therefore causes a proportional increase in fluorescence due to the breakdown of the probe and release of the reporter. An additional advantage of real-time PCR is its quantitative nature. Each positive sample is given a Ct (defined as the PCR cycle in which the level of fluorescence has crossed the threshold). By running a series of samples containing known quantities of target RNA or DNA, a standard curve can be created that correlates sample Ct to the initial quantity of target RNA or DNA in a sample. Unknown samples are then tested and their Ct values are compared with the standard curve to determine the initial quantity of target RNA/DNA present in the sample. Alternatively, relative quantification can be assessed based on internal reference genes to determine fold-differences in expression of the target gene.

Amplified nucleic acid may also be detected by labelling one of the primers (e.g. forward or reverse) primer used in the amplification process. Many methods for the detection of allelic variation are described in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

By detecting the presence, or quantitating the amount of, target allele, the present invention can be used to draw conclusions regarding the subject from which the test sample is taken. For example, target allele that is detected in the present invention may be an allele that is predictive of a diagnosis and/or a prognosis of a subject from which the sample is taken. Accordingly, by detecting and/or quantifying the amplified cDNA of the target allele, an assessment can be made as to whether the subject is likely to have a particular disease. Alternatively, by detecting and/or quantifying the amplified cDNA of the target allele, an assessment of the likely prognosis of the subject can be made. An example of such a prognosis is the prediction of the likelihood of a disease recurrence. An example of such a disease is cancer.

In a further embodiment of the present invention, the target allele that is detected may be correlated with the likelihood of success of a particular drug treatment. Great inroads have been made in the area of personalised medicines and there are now multiple drug treatments that are specific sub-populations that have a common genetic trait such as a specific mutant allele. Accordingly, the present invention can be used to detect for the presence of or quantify the amount of target allele in a sample derived from a subject, and the detection of, or amount of, target allele expression can be used in determining the treatment of the subject with a drug associated with modifying (e.g. inhibiting) the expression of the target allele or the activity of the protein expressed by the target allele.

An example is the treatment of erlotinib seen in non-small cell lung cancer (NSCLC) patients with EGFR mutations. One such mutation is EGFR L858R. Accordingly, in a further embodiment, the method of the present invention may be used to analyze a patient sample for expression (or absence of expression) of a EGFR mutant allele or to quantify the level of expression of EGFR mutant allele in a patient sample. In a further aspect of this embodiment, the patient has, or is suspected of having NSCLC. In a yet further aspect, the present invention can be used to detect for the expression of a EGFR mutant allele in a sample derived from a patient and the detection of, or level of EGFR mutant allele can be used to decide, or aid, in the decision as to whether to administer erlotinib or a drug capable of modifying the activity or expression of EGFR. The EGFR mutant allele may be a L858 mutant allele (i.e. where the L residue at position 858 is replaced by a different (mutant) residue), preferably the EGFR L858R mutant allele.

Two recent examples of the trend towards targeted therapies are crizotinib and vemurafenib for patients with EML4-ALK fusions and BRAF V600 mutations, respectively.

Vemurafenib which has been approved by the Food and Drug Administration to treat patients with metastatic melanoma who have a BRAF V600 mutation, such asV600E, V600K, V600D or V600R mutation. The BRAF protein is normally involved in regulatory cell growth, but is mutated in about half of patients with late-stage melanomas. Vemurafenib is a BRAF inhibitor that is able to block the function of the V600E-mutated BRAF protein from driving the proliferation of cancer cells.

Accordingly, in one embodiment, the method of the present invention may be used to analyze a patient sample for expression of a BRAF mutant allele or to quantify the level of expression of BRAF mutant allele. In a further aspect of this embodiment, the to patient has, or is suspected of having metastatic melanoma. In a yet further aspect, the present invention can be used to detect for the expression of a BRAF mutant allele in a sample derived from a patient and the detection of, or level of BRAF mutant allele can be used to decide, or aid, in the decision as to whether to administer a specific therapy or a drug capable of modifying the activity or expression of BRAF. Preferably, the BRAF mutant allele is a V600 mutant allele (i.e. where the V residue at position 600 is replaced by a different (mutant) residue). V600 mutant alleles may include V600E, V600K, V600D or V600R allele, more preferably the V600E or V600K allele.

The protein sequence encoded by the human BRAF gene is set out below (SEQ ID NO: 10):

(SEQ ID NO: 10)
MAALSGGGGGGAEPGQALFNGDMEPEAGAGAGAAASSAADPAIP
EEVWNIKQMIKLTQEHIEALLDKFGGEHNPPSIYLEAYEEYTSKLDALQQREQQLLES
LGNGTDFSVSSSASMDTVTSSSSSSLSVLPSSLSVFQNPTDVARSNPKSPQKPIVRVF
LPNKQRTVVPARCGVTVRDSLKKALMMRGLIPECCAVYRIQDGEKKPIGWDTDISWLT
GEELHVEVLENVPLTTHNFVRKTFFTLAFCDFCRKLLFQGFRCQTCGYKFHQRCSTEV
PLMCVNYDQLDLLFVSKFFEHHPIPQEEASLAETALTSGSSPSAPASDSIGPQILTSP
SPSKSIPIPQPFRPADEDHRNQFGQRDRSSSAPNVHINTIEPVNIDDLIRDQGFRGDG
GSTTGLSATPPASLPGSLTNVKALQKSPGPQRERKSSSSSEDRNRMKTLGRRDSSDDW
EIPDGQITVGQRIGSGSFGTVYKGKWHGDVAVKMLNVTAPTPQQLQAFKNEVGVLRKT
RHVNILLFMGYSTKPQLAIVTQWCEGSSLYHHLHIIETKFEMIKLIDIARQTAQGMDY
LHAKSIIHRDLKSNNIFLHEDLTVKIGDFGLATVKSRWSGSHQFEQLSGSILWMAPEV
IRMQDKNPYSFQSDVYAFGIVLYELMTGQLPYSNINNRDQIIFMVGRGYLSPDLSKVR
SNCPKAMKRLMAECLKKKRDERPLFPQILASIELLAEDFSLYACASPKTPIQAGGYGAFPVH

In a further embodiment of the present invention, the target allele that is detected may be correlated with resistance to particular drug treatment. For example crizotinib can be used for patients with EML4-ALK fusions, particularly patients with NSCLC, anaplastic large cell lymphoma, neuroblastoma, or other advanced tumors. It has been identified that the ALK C1156Y mutation in the tyrosine kinase domain confers resistance to crizotinib. Accordingly, the method of the present invention may be used to analyze a patient sample for the expression of an ALK mutant allele or to quantify the level of expression of an ALK mutant allele. In a further aspect of this embodiment, the patients have, or are suspected of having NSCLC, anaplastic large cell lymphoma, or neuroblastoma, or other advanced tumors. In a yet further aspect, the present invention can be used to detect for the expression of an ALK mutant allele in a sample derived from a patient and the detection of, or level of ALK mutant allele can be used to decide, or aid, in the decision as to whether to administer crizotinib or a drug capable of modifying the activity or expression of anaplastic lymphoma kinase (ALK). The ALK mutant allele may be an ALK C1156 mutant allele (i.e. where the C residue at position 1156 is replaced by a different (mutant) residue), preferably the ALK C1156Y allele.

A further example is the resistance to EGFR tyrosine kinase inhibitors (EGFR-TKIs) seen in patients with lung cancer. A secondary point mutation that substitutes methionine in place of threonine at amino acid position 790 (T790M) is a molecular mechanism that produces a drug-resistant variant of the targeted kinase. Accordingly, in a further embodiment, the method of the present invention may be used to analyze a patient sample for expression of an EGFR mutant allele or to quantify the level of expression of an EGFR mutant allele. In a further aspect of this embodiment, the patient has, or is suspected of having lung cancer. In a yet further aspect, the present invention can be used to detect for the expression of an EGFR mutant allele in a sample derived from a patient and the detection of, or level of EGFR mutant allele can be used to decide, or aid, in the decision as to whether to administer an EGFR tyrosine kinase inhibitor. The EGFR mutant allele may be an EGFR T790 mutant allele (i.e. where the T residue at position 790 is replaced by a different (mutant) residue), preferably the EGFR T790M allele.

Another example is the resistance to BCR-ABL inhibitors (e.g. imatinib) seen in patients with a T315I mutation in the ABL gene. It can be caused by a single cytosine to thymine (C->T) base pair substitution at position 944 of the Abl gene (codon ‘315’ of the Abl protein) sequence resulting in amino acid (T)hreonine being substituted by (I)soleucine at that position—thus ‘T315I’.

Accordingly, in a further embodiment, the method of the present invention may be used to analyze a patient sample for expression of an ABL mutant allele or to quantify the level of expression of an ABL mutant allele. In a further aspect of this embodiment, the patients have, or are suspected of having chronic myelogenous leukemia. In a yet further aspect, the present invention can be used to detect for the expression of an ABL mutant allele in a sample derived from a patient and the detection of, or level of ABL mutant allele can be used to decide, or aid, in the decision as to whether to administer imatinib or a BCR-ABL inhibitor. The ABL mutant allele may be a T315 mutant allele (i.e. where the T residue at position 315 is replaced by a different (mutant) residue), preferably the ABL T315I mutant allele.

The ABL protein sequence (also known as ABL1) is shown below (SEQ ID NO: 11), with amino acid at position 315 highlighted:

(SEQ ID NO: 11)
MLEICLKLVGCKSKKGLSSSSSCYLEEALQRPVASD
FEPQGLSEAARWNSKENLLAGPSENDPNLFVALYDFVASGDNTLSITKGE
KLRVLGYNHNGEWCEAQTKNGQGWVPSNYITPVNSLEKHSWYHGPVSRNA
AEYLLSSGINGSFLVRESESSPGQRSISLRYEGRVYHYRINTASDGKLYV
SSESRFNTLAELVHHHSTVADGLITTLHYPAPKRNKPTVYGVSPNYDKWE
MERTDITMKHKLGGGQYGEVYEGVWKKYSLTVAVKTLKEDTMEVEEFLKE
AAVMKEIKHPNLVQLLGVCTREPPFYIITEFMTYGNLLDYLRECNRQEVN
AVVLLYMATQISSAMEYLEKKNFIHRDLAARNCLVGENHLVKVADEGLSR
LMTGDTYTAHAGAKFPIKWTAPESLAYNKFSIKSDVWAFGVLLWEIATYG
MSPYPGIDLSQVYELLEKDYRMERPEGCPEKVYELMRACWQWNPSDRPSF
AEIHQAFETMFQESSISDEVEKELGKQGVRGAVSTLLQAPELPTKTRTSR
RAAEHRDTTDVPEMPHSKGQGESDPLDHEPAVSPLLPRKERGPPEGGLNE
DERLLPKDKKTNLFSALIKKKKKTAPTPPKRSSSFREMDGQPERRGAGEE
EGRDISNGALAFTPLDTADPAKSPKPSNGAGVPNGALRESGGSGFRSPHL
WKKSSTLTSSRLATGEEEGGGSSSKRFLRSCSASCVPHGAKDTEWRSVTL
PRDLQSTGRQFDSSTFGGHKSEKPALPRKRAGENRSDQVTRGTVTPPPRL
VKKNEEAADEVFKDIMESSPGSSPPNLTPKPLRRQVTVAPASGLPHKEEA
GKGSALGTPAAAEPVTPTSKAGSGAPGGTSKGPAEESRVRRHKHSSESPG
RDKGKLSRLKPAPPPPPAASAGKAGGKPSQSPSQEAAGEAVLGAKTKATS
LVDAVNSDAAKPSQPGEGLKKPVLPATPKPQSAKPSGTPISPAPVPSTLP
SASSALAGDQPSSTAFIPLISTRVSLRKTRQPPERIASGAITKGVVLDST
EALCLAISRNSEQMASHSAVLEAGKNLYTFCVSYVDSIQQMRNKFAFREA
INKLENNLRELQICPATAGSGPAATQDFSKLLSSVKEISDIVQR

The corresponding ABL mRNA transcript (shown here in cDNA format where base u is replaced by t) is set out below (SEQ ID NO:12), with the mutation base at position 947 highlighted in bold (where replacement of c by t (u in the case of mRNA) gives rise to the T315I mutation).

(SEQ ID NO: 12)
aaaatgttggagatctgcctgaagctggtgggctgcaaatccaagaaggggctgtcctcgtcctccagctgt
tatctggaagaagcccttcagcggccagtagcatctgactttgagcctcagggtctgagtgaagccgctcgt
tggaactccaaggaaaaccttctcgctggacccagtgaaaatgaccccaaccttttcgttgcactgtatgat
tttgtggccagtggagataacactctaagcataactaaaggtgaaaagctccgggtcttaggctataatcac
aatggggaatggtgtgaagcccaaaccaaaaatggccaaggctgggtcccaagcaactacatcacgccagtc
aacagtctggagaaacactcctggtaccatgggcctgtgtcccgcaatgccgctgagtatctgctgagcagc
gggatcaatggcagcttcttggtgcgtgagagtgagagcagtcctggccagaggtccatctcgctgagatac
gaagggagggtgtaccattacaggatcaacactgcttctgatggcaagctctacgtctcctccgagagccgc
ttcaacaccctggccgagttggttcatcatcattcaacggtggccgacgggctcatcaccacgctccattat
ccagccccaaagcgcaacaagcccactgtctatggtgtgtcccccaactacgacaagtgggagatggaacgc
acggacatcaccatgaagcacaagctgggcgggggccagtacggggaggtgtacgagggcgtgtggaagaaa
tacagcctgacggtggccgtgaagaccttgaaggaggacaccatggaggtggaagagttcttgaaagaagct
gcagtcatgaaagagatcaaacaccctaacctggtgcagctccttggggtctgcacccgggagcccccgttc
tatatcatcaCtgagttcatgacctacgggaacctcctggactacctgagggagtgcaaccggcaggaggt
gaacgccgtggtgctgctgtacatggccactcagatctcgtcagccatggagtacctggagaagaaaaactt
catccacagagatcttgctgcccgaaactgcctggtaggggagaaccacttggtgaaggtagctgattttgg
cctgagcaggttgatgacaggggacacctacacagcccatgctggagccaagttccccatcaaatggactgc
acccgagagcctggcctacaacaagttctccatcaagtccgacgtctgggcatttggagtattgctttggga
aattgctacctatggcatgtccccttacccgggaattgacctgtcccaggtgtatgagctgctagagaagga
ctaccgcatggagcgcccagaaggctgcccagagaaggtctatgaactcatgcgagcatgttggcagtggaa
tccctctgaccggccctcctttgctgaaatccaccaagcctttgaaacaatgttccaggaatccagtatctc
agacgaagtggaaaaggagctggggaaacaaggcgtccgtggggctgtgagtaccttgctgcaggccccaga
gctgcccaccaagacgaggacctccaggagagctgcagagcacagagacaccactgacgtgcctgagatgcc
tcactccaagggccagggagagagcgatcctctggaccatgagcctgccgtgtctccattgctccctcgaaa
agagcgaggtcccccggagggcggcctgaatgaagatgagcgccttctccccaaagacaaaaagaccaactt
gttcagcgccttgatcaagaagaagaagaagacagccccaacccctcccaaacgcagcagctccttccggga
gatggacggccagccggagcgcagaggggccggcgaggaagagggccgagacatcagcaacggggcactggc
tttcacccccttggacacagctgacccagccaagtccccaaagcccagcaatggggctggggtccccaatgg
agccctccgggagtccgggggctcaggcttccggtctccccacctgtggaagaagtccagcacgctgaccag
cagccgcctagccaccggcgaggaggagggcggtggcagctccagcaagcgcttcctgcgctcttgctccgc
ctcctgcgttccccatggggccaaggacacggagtggaggtcagtcacgctgcctcgggacttgcagtccac
gggaagacagtttgactcgtccacatttggagggcacaaaagtgagaagccggctctgcctcggaagagggc
aggggagaacaggtctgaccaggtgacccgaggcacagtaacgcctccccccaggctggtgaaaaagaatga
ggaagctgctgatgaggtcttcaaagacatcatggagtccagcccgggctccagcccgcccaacctgactcc
aaaacccctccggcggcaggtcaccgtggcccctgcctcgggcctcccccacaaggaagaagctggaaaggg
cagtgccttagggacccctgctgcagctgagccagtgacccccaccagcaaagcaggctcaggtgcaccagg
gggcaccagcaagggccccgccgaggagtccagagtgaggaggcacaagcactcctctgagtcgccagggag
ggacaaggggaaattgtccaggctcaaacctgccccgccgcccccaccagcagcctctgcagggaaggctgg
aggaaagccctcgcagagcccgagccaggaggcggccggggaggcagtcctgggcgcaaagacaaaagccac
gagtctggttgatgctgtgaacagtgacgctgccaagcccagccagccgggagagggcctcaaaaagcccgt
gctcccggccactccaaagccacagtccgccaagccgtcggggacccccatcagcccagcccccgttccctc
cacgttgccatcagcatcctcggccctggcaggggaccagccgtcttccaccgccttcatccctctcatatc
aacccgagtgtctcttcggaaaacccgccagcctccagagcggatcgccagcggcgccatcaccaagggcgt
ggtcctggacagcaccgaggcgctgtgcctcgccatctctaggaactccgagcagatggccagccacagcgc
agtgctggaggccggcaaaaacctctacacgttctgcgtgagctatgtggattccatccagcaaatgaggaa
caagtttgccttccgagaggccatcaacaaactggagaataatctccgggagcttcagatctgcccggcgac
agcaggcagtggtccagcggccactcaggacttcagcaagctcctcagttcggtgaaggaaatcagtgacat
agtgcagaggtagcagcagtcaggggtcaggtgtcaggcccgtcggagctgcctgcagcacatgcgggctcg
cccatacccgtgacagtggctgacaagggactagtgagtcagcaccttggcccaggagctctgcgccaggca
gagctgagggccctgtggagtccagctctactacctacgtttgcaccgcctgccctcccgcaccttcctcct
ccccgctccgtctctgtcctcgaattttatctgtggagttcctgctccgtggactgcagtcggcatgccagg
acccgccagccccgctcccacctagtgccccagactgagctctccaggccaggtgggaacggctgatgtgga
ctgtctttttcatttttttctctctggagcccctcctcccccggctgggcctccttcttccacttctccaag
aatggaagcctgaactgaggccttgtgtgtcaggccctctgcctgcactccctggccttgcccgtcgtgtgc
tgaagacatgtttcaagaaccgcatttcgggaagggcatgcacgggcatgcacacggctggtcactctgccc
tctgctgctgcccggggtggggtgcactcgccatttcctcacgtgcaggacagctcttgatttgggtggaaa
acagggtgctaaagccaaccagcctttgggtcctgggcaggtgggagctgaaaaggatcgaggcatggggca
tgtcctttccatctgtccacatccccagagcccagctcttgctctcttgtgacgtgcactgtgaatcctggc
aagaaagcttgagtctcaagggtggcaggtcactgtcactgccgacatccctcccccagcagaatggaggca
ggggacaagggaggcagtggctagtggggtgaacagctggtgccaaatagccccagactgggcccaggcagg
tctgcaagggcccagagtgaaccgtcctttcacacatctgggtgccctgaaagggcccttcccctcccccac
tcctctaagacaaagtagattcttacaaggccctttcctttggaacaagacagccttcacttttctgagttc
ttgaagcatttcaaagccctgcctctgtgtagccgccctgagagagaatagagctgccactgggcacctgcg
cacaggtgggaggaaagggcctggccagtcctggtcctggctgcactcttgaactgggcgaatgtcttattt
aattaccgtgagtgacatagcctcatgttctgtgggggtcatcagggagggttaggaaaaccacaaacggag
cccctgaaagcctcacgtatttcacagagcacgcctgccatcttctccccgaggctgccccaggccggagcc
cagatacgggggctgtgactctgggcagggacccggggtctcctggaccttgacagagcagctaactccgag
agcagtgggcaggtggccgcccctgaggcttcacgccgggagaagccaccttcccaccccttcataccgcct
cgtgccagcagcctcgcacaggccctagctttacgctcatcacctaaacttgtactttatttttctgataga
aatggtttcctctggatcgttttatgcggttcttacagcacatcacctctttgcccccgacggctgtgacgc
agccggagggaggcactagtcaccgacagcggccttgaagacagagcaaagcgcccacccaggtcccccgac
tgcctgtctccatgaggtactggtcccttccttttgttaacgtgatgtgccactatattttacacgtatctc
ttggtatgcatcttttatagacgctcttttctaagtggcgtgtgcatagcgtcctgccctgccccctcgggg
gcctgtggtggctccccctctgcttctcggggtccagtgcattttgtttctgtatatgattctctgtggttt
tttttgaatccaaatctgtcctctgtagtattttttaaataaatcagtgtttacattagaa

Detection of the ABL T315 mutation is particularly important in patients expressing the fusion gene BCR-ABL. In patients with e.g. chronic myelogenous leukemia (CML), the ABL gene is activated by being translocated within the BCR (breakpoint cluster region) gene on chromosome 22. This fusion gene, BCR-ABL, encodes a tyrosine kinase that allows cells to proliferate without being regulated by cytokines, which in turn allows the cell to become cancerous.

As highlighted above, the presence of the ABL T315I mutation in the BCR-ABL fusion protein is significant because patients with this mutation show resistance to tyrosine kinase inhibitors. The substitution eliminates a critical oxygen molecule needed for hydrogen bonding between imatinib and the Abl kinase, and also creates steric hindrance to the binding of most tyrosine kinase inhibitors.

An example BCR-ABL fusion protein (the b2a2 protein) sequence is set out below (SEQ ID NO:13). The position of the ABL amino acid corresponding to the ABLT315I mutation is shown in bold and underlined. The amino acid showing the fusion junction between the BCR and ABL is also shown in bold.

(SEQ ID NO:13)
MVDPVGFAEAWKAQFPDSEPPRMELRSVGDIEQELERCKASIRRLEQEVNQERFRMIYLQTLLAKEKKSYDR
QRWGFRRAAQAPDGASEPRASASRPQPAPADGADPPPAEEPEARPDGEGSPGKARPGTARRPGAAASGERDD
RGPPASVAALRSNFERIRKGHGQPGADAEKPFYVNVEFHHERGLVKVNDKEVSDRISSLGSQAMQMERKKSQ
HGAGSSVGDASRPPYRGRSSESSCGVDGDYEDAELNPRFLKDNLIDANGGSRPPWPPLEYQPYQSIYVGGMM
EGEGKGPLLRSQSTSEQEKRLTWPRRSYSPRSFEDCGGGYTPDCSSNENLTSSEEDFSSGQSSRVSPSPTTY
RMFRDKSRSPSQNSQQSFDSSSPPTPQCHKRHRHCPVVVSEATIVGVRKTGQIWPNDGEGAFHGDADGSFGT
PPGYGCAADRAEEQRRHQDGLPYIDDSPSSSPHLSSKGRGSRDALVSGALESTKASELDLEKGLEMRKWVLS
GILASEETYLSHLEALLLPMKPLKAAATTSQPVLTSQQIETIFFKVPELYEIHKEFYDGLFPRVQQWSHQQR
VGDLFQKLASQLGVYRAFVDNYGVAMEMAEKCCQANAQFAEISENLRARSNKDAKDPTTKNSLETLLYKPVD
RVTRSTLVLHDLLKHTPASHPDHPLLQDALRISQNFLSSINEEITPRRQSMTVKKGEHRQLLKDSFMVELVE
GARKLRHVFLFTDLLLCTKLKKQSGGKTQQYDCKWYIPLTDLSFQMVDELEAVPNIPLVPDEELDALKIKIS
QIKNDIQREKRANKGSKATERLKKKLSEQESLLLLMSPSMAFRVHSRNGKSYTFLISSDYERAEWRENIREQ

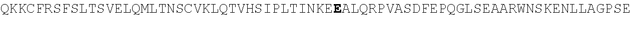

NDPNLFVALYDFVASGDNTLSITKGEKLRVLGYNHNGEWCEAQTKNGQGWVPSNYITPVNSLEKHSWYHGPV
SRNAAEYLLSSGINGSFLVRESESSPGQRSISLRYEGRVYHYRINTASDGKLYVSSESRFNTLAELVHHHST
VADGLITTLHYPAPKRNKPTVYGVSPNYDKWEMERTDITMKHKLGGGQYGEVYEGVWKKYSLTVAVKTLKED

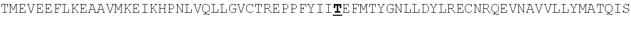

SAMEYLEKKNFIHRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNPSDRPSFAEIHQA
FETMFQESSISDEVEKELGKQGVRGAVSTLLQAPELPTKTRTSRRAAEHRDTTDVPEMPHSKGQGESDPLDH
EPAVSPLLPRKERGPPEGGLNEDERLLPKDKKTNLFSALIKKKKKTAPTPPKRSSSFREMDGQPERRGAGEE
EGRDISNGALAFTPLDTADPAKSPKPSNGAGVPNGALRESGGSGFRSPHLWKKSSTLTSSRLATGEEEGGGS
SSKRFLRSCSASCVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGSALGTPAAAEPVT
PTSKAGSGAPGGTSKGPAEESRVRRHKHSSESPGRDKGKLSRLKPAPPPPPAASAGKAGGKPSQSPSQEAAG
EAVLGAKTKATSLVDAVNSDAAKPSQPGEGLKKPVLPATPKPQSAKPSGTPISPAPVPSTLPSASSALAGDQ
PSSTAFIPLISTRVSLRKTRQPPERIASGAITKGVVLDSTEALCLAISRNSEQMASHSAVLEAGKNLYTFCV
SYVDSIQQMRNKFAFREAINKLENNLRELQICPATAGSGPAATQDFSKLLSSVKEISDIVQR

The corresponding mRNA sequence (shown here in cDNA format where base u is replaced by t) is set out below (SEQ ID NO:14), with the position of the base that gives rise to the T315I mutation when the base changes from c to t is highlighted in bold and underlined and the base showing the position of the ABL-BCR fusion junction shown in bold).

(SEQ ID NO: 14)
ggggggagggtggcggctcgatgggggagccgcctccagggggcccccccgccctgtgcccacggcgcggcc
cctttaagaggcccgcctggctccgtcatccgcgccgcggccacctccccccggccctccccttcctgcggc
gcagagtgcgggccgggcgggagtgcggcgagagccggctggctgagcttagcgtccgaggaggcggcggcg
gcggcggcggcacggcggcggcggggctgtggggcggtgcggaagcgagaggcgaggagcgcgcgggccgtg
gccagagtctggcggcggcctggcggagcggagagcagcgcccgcgcctcgccgtgcggaggagccccgcac
acaatagcggcgcgcgcagcccgcgcccttccccccggcgcgccccgccccgcgcgccgagcgccccgctcc
gcctcacctgccaccagggagtgggcgggcattgttcgccgccgccgccgccgcgcgggccatgggggccgc
ccggcgcccggggccgggctggcgaggcgccgcgccgccgctgagacgggccccgcgcgcagcccggcggcg
caggtaaggccggccgcgccatggtggacccggtgggcttcgcggaggcgtggaaggcgcagttcccggact
cagagcccccgcgcatggagctgcgctcagtgggcgacatcgagcaggagctggagcgctgcaaggcctcca
ttcggcgcctggagcaggaggtgaaccaggagcgcttccgcatgatctacctgcagacgttgctggccaagg
aaaagaagagctatgaccggcagcgatggggcttccggcgcgcggcgcaggcccccgacggcgcctccgagc
cccgagcgtccgcgtcgcgcccgcagccagcgcccgccgacggagccgacccgccgcccgccgaggagcccg
aggcccggcccgacggcgagggttctccgggtaaggccaggcccgggaccgcccgcaggcccggggcagccg
cgtcgggggaacgggacgaccggggaccccccgccagcgtggcggcgctcaggtccaacttcgagcggatcc
gcaagggccatggccagcccggggcggacgccgagaagcccttctacgtgaacgtcgagtttcaccacgagc
gcggcctggtgaaggtcaacgacaaagaggtgtcggaccgcatcagctccctgggcagccaggccatgcaga
tggagcgcaaaaagtcccagcacggcgcgggctcgagcgtgggggatgcatccaggcccccttaccggggac
gctcctcggagagcagctgcggcgtcgacggcgactacgaggacgccgagttgaacccccgcttcctgaagg
acaacctgatcgacgccaatggcggtagcaggcccccttggccgcccctggagtaccagccctaccagagca
tctacgtcgggggcatgatggaaggggagggcaagggcccgctcctgcgcagccagagcacctctgagcagg
agaagcgccttacctggccccgcaggtcctactccccccggagttttgaggattgcggaggcggctataccc
cggactgcagctccaatgagaacctcacctccagcgaggaggacttctcctctggccagtccagccgcgtgt
ccccaagccccaccacctaccgcatgttccgggacaaaagccgctctccctcgcagaactcgcaacagtcct
tcgacagcagcagtccccccacgccgcagtgccataagcggcaccggcactgcccggttgtcgtgtccgagg
ccaccatcgtgggcgtccgcaagaccgggcagatctggcccaacgatggcgagggcgccttccatggagacg
cagatggctcgttcggaacaccacctggatacggctgcgctgcagaccgggcagaggagcagcgccggcacc
aagatgggctgccctacattgatgactcgccctcctcatcgccccacctcagcagcaagggcaggggcagcc
gggatgcgctggtctcgggagccctggagtccactaaagcgagtgagctggacttggaaaagggcttggaga
tgagaaaatgggtcctgtcgggaatcctggctagcgaggagacttacctgagccacctggaggcactgctgc
tgcccatgaagcctttgaaagccgctgccaccacctctcagccggtgctgacgagtcagcagatcgagacca
tcttcttcaaagtgcctgagctctacgagatccacaaggagttctatgatgggctcttcccccgcgtgcagc
agtggagccaccagcagcgggtgggcgacctcttccagaagctggccagccagctgggtgtgtaccgggcct
tcgtggacaactacggagttgccatggaaatggctgagaagtgctgtcaggccaatgctcagtttgcagaaa
tctccgagaacctgagagccagaagcaacaaagatgccaaggatccaacgaccaagaactctctggaaactc
tgctctacaagcctgtggaccgtgtgacgaggagcacgctggtcctccatgacttgctgaagcacactcctg
ccagccaccctgaccaccccttgctgcaggacgccctccgcatctcacagaacttcctgtccagcatcaatg
aggagatcacaccccgacggcagtccatgacggtgaagaagggagagcaccggcagctgctgaaggacagct
tcatggtggagctggtggagggggcccgcaagctgcgccacgtcttcctgttcaccgacctgcttctctgca
ccaagctcaagaagcagagcggaggcaaaacgcagcagtatgactgcaaatggtacattccgctcacggatc
tcagcttccagatggtggatgaactggaggcagtgcccaacatccccctggtgcccgatgaggagctggacg
ctttgaagatcaagatctcccagatcaagaatgacatccagagagagaagagggcgaacaagggcagcaagg
ctacggagaggctgaagaagaagctgtcggagcaggagtcactgctgctgcttatgtctcccagcatggcct
tcagggtgcacagccgcaacggcaagagttacacgttcctgatctcctctgactatgagcgtgcagagtgga
gggagaacatccgggagcagcagaagaagtgtttcagaagcttctccctgacatccgtggagctgcagatgc

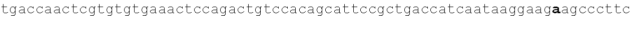

agcggccagtagcatctgactttgagcctcagggtctgagtgaagccgctcgttggaactccaaggaaaacc
ttctcgctggacccagtgaaaatgaccccaaccttttcgttgcactgtatgattttgtggccagtggagata
acactctaagcataactaaaggtgaaaagctccgggtcttaggctataatcacaatggggaatggtgtgaag
cccaaaccaaaaatggccaaggctgggtcccaagcaactacatcacgccagtcaacagtctggagaaacact
cctggtaccatgggcctgtgtcccgcaatgccgctgagtatctgctgagcagcgggatcaatggcagcttct
tggtgcgtgagagtgagagcagtcctggccagaggtccatctcgctgagatacgaagggagggtgtaccatt
acaggatcaacactgcttctgatggcaagctctacgtctcctccgagagccgcttcaacaccctggccgagt
tggttcatcatcattcaacggtggccgacgggctcatcaccacgctccattatccagccccaaagcgcaaca
agcccactgtctatggtgtgtcccccaactacgacaagtgggagatggaacgcacggacatcaccatgaagc
acaagctgggcgggggccagtacggggaggtgtacgagggcgtgtggaagaaatacagcctgacggtggccg
tgaagaccttgaaggaggacaccatggaggtggaagagttcttgaaagaagctgcagtcatgaaagagatca

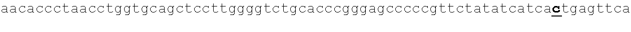

tgacctacgggaacctcctggactacctgagggagtgcaaccggcaggaggtgaacgccgtggtgctgctgt
acatggccactcagatctcgtcagccatggagtacctggagaagaaaaacttcatccacagagatcttgctg
cccgaaactgcctggtaggggagaaccacttggtgaaggtagctgattttggcctgagcaggttgatgacag
gggacacctacacagcccatgctggagccaagttccccatcaaatggactgcacccgagagcctggcctaca
acaagttctccatcaagtccgacgtctgggcatttggagtattgctttgggaaattgctacctatggcatgt
ccccttacccgggaattgacctgtcccaggtgtatgagctgctagagaaggactaccgcatggagcgcccag
aaggctgcccagagaaggtctatgaactcatgcgagcatgttggcagtggaatccctctgaccggccctcct
ttgctgaaatccaccaagcctttgaaacaatgttccaggaatccagtatctcagacgaagtggaaaaggagc
tggggaaacaaggcgtccgtggggctgtgagtaccttgctgcaggccccagagctgcccaccaagacgagga
cctccaggagagctgcagagcacagagacaccactgacgtgcctgagatgcctcactccaagggccagggag
agagcgatcctctggaccatgagcctgccgtgtctccattgctccctcgaaaagagcgaggtcccccggagg
gcggcctgaatgaagatgagcgccttctccccaaagacaaaaagaccaacttgttcagcgccttgatcaaga
agaagaagaagacagccccaacccctcccaaacgcagcagctccttccgggagatggacggccagccggagc
gcagaggggccggcgaggaagagggccgagacatcagcaacggggcactggctttcacccccttggacacag
ctgacccagccaagtccccaaagcccagcaatggggctggggtccccaatggagccctccgggagtccgggg
gctcaggcttccggtctccccacctgtggaagaagtccagcacgctgaccagcagccgcctagccaccggcg
aggaggagggcggtggcagctccagcaagcgcttcctgcgctcttgctccgcctcctgcgttccccatgggg
ccaaggacacggagtggaggtcagtcacgctgcctcgggacttgcagtccacgggaagacagtttgactcgt
ccacatttggagggcacaaaagtgagaagccggctctgcctcggaagagggcaggggagaacaggtctgacc
aggtgacccgaggcacagtaacgcctccccccaggctggtgaaaaagaatgaggaagctgctgatgaggtct
tcaaagacatcatggagtccagcccgggctccagcccgcccaacctgactccaaaacccctccggcggcagg
tcaccgtggcccctgcctcgggcctcccccacaaggaagaagctggaaagggcagtgccttagggacccctg
ctgcagctgagccagtgacccccaccagcaaagcaggctcaggtgcaccagggggcaccagcaagggccccg
ccgaggagtccagagtgaggaggcacaagcactcctctgagtcgccagggagggacaaggggaaattgtcca
ggctcaaacctgccccgccgcccccaccagcagcctctgcagggaaggctggaggaaagccctcgcagagcc
cgagccaggaggcggccggggaggcagtcctgggcgcaaagacaaaagccacgagtctggttgatgctgtga
acagtgacgctgccaagcccagccagccgggagagggcctcaaaaagcccgtgctcccggccactccaaagc
cacagtccgccaagccgtcggggacccccatcagcccagcccccgttccctccacgttgccatcagcatcct
cggccctggcaggggaccagccgtcttccaccgccttcatccctctcatatcaacccgagtgtctcttcgga
aaacccgccagcctccagagcggatcgccagcggcgccatcaccaagggcgtggtcctggacagcaccgagg
cgctgtgcctcgccatctctaggaactccgagcagatggccagccacagcgcagtgctggaggccggcaaaa
acctctacacgttctgcgtgagctatgtggattccatccagcaaatgaggaacaagtttgccttccgagagg
ccatcaacaaactggagaataatctccgggagcttcagatctgcccggcgacagcaggcagtggtccagcgg
ccactcaggacttcagcaagctcctcagttcggtgaaggaaatcagtgacatagtgcagaggtagcagcagt
caggggtcaggtgtcaggcccgtcggagctgcctgcagcacatgcgggctcgcccatacccgtgacagtggc
tgacaagggactagtgagtcagcaccttggcccaggagctctgcgccaggcagagctgagggccctgtggag
tccagctctactacctacgtttgcaccgcctgccctcccgcaccttcctcctccccgctccgtctctgtcct
cgaattttatctgtggagttcctgctccgtggactgcagtcggcatgccaggacccgccagccccgctccca
cctagtgccccagactgagctctccaggccaggtgggaacggctgatgtggactgtctttttcatttttttc
tctctggagcccctcctcccccggctgggcctccttcttccacttctccaagaatggaagcctgaactgagg
ccttgtgtgtcaggccctctgcctgcactccctggccttgcccgtcgtgtgctgaagacatgtttcaagaac
cgcatttcgggaagggcatgcacgggcatgcacacggctggtcactctgccctctgctgctgcccggggtgg
ggtgcactcgccatttcrctcacgtgcaggacagctcttgatttgggtggaaaacagggtgctaaagccaacc
agcctttgggtcctgggcaggtgggagctgaaaaggatcgaggcatggggcatgtcctttccatctgtccac
atccccagagcccagctcttgctctcttgtgacgtgcactgtgaatcctggcaagaaagcttgagtctcaag
ggtggcaggtcactgtcactgccgacatccctcccccagcagaatggaggcaggggacaagggaggcagtgg
ctagtggggtgaacagctggtgccaaatagccccagactgggcccaggcaggtctgcaagggcccagagtga
accgtcctttcacacatctgggtgccctgaaagggcccttcccctcccccactcctctaagacaaagtagat
tcttacaaggccctttcctttggaacaagacagccttcacttttctgagttcttgaagcatttcaaagccct
gcctctgtgtagccgccctgagagagaatagagctgccactgggcacctgcgcacaggtgggaggaaagggc
ctggccagtcctggtcctggctgcactcttgaactgggcgaatgtcttatttaattaccgtgagtgacatag
cctcatgttctgtgggggtcatcagggagggttaggaaaaccacaaacggagcccctgaaagcctcacgtat
ttcacagagcacgcctgccatcttctccccgaggctgccccaggccggagcccagatacgggggctgtgact
ctgggcagggacccggggtctcctggaccttgacagagcagctaactccgagagcagtgggcaggtggccgc
ccctgaggcttcacgccgggagaagccaccttcccaccccttcataccgcctcgtgccagcagcctcgcaca
ggccctagctttacgctcatcacctaaacttgtactttatttttctgatagaaatggtttcctctggatcgt
tttatgcggttcttacagcacatcacctctttgcccccgacggctgtgacgcagccggagggaggcactagt
caccgacagcggccttgaagacagagcaaagcgcccacccaggtcccccgactgcctgtctccatgaggtac
tggtcccttccttttgttaacgtgatgtgccactatattttacacgtatctcttggtatgcatcttttatag
acgctcttttctaagtggcgtgtgcatagcgtcctgccctgccccctcgggggcctgtggtggctccccctc
tgcttctcggggtccagtgcattttgtttctgtatatgattctctgtggttttttttgaatccaaatctgtc
ctctgtagtattttttaaataaatcagtgtttacattagaa

Accordingly, in a further aspect, the present invention provides a method of detecting for the presence of a mutation in the ABL gene coding for the ABL T315I mutation, the method comprising:
a) providing a sample comprising an mRNA transcript encoding the ABL T315I mutation;
b) performing a reverse-transcription reaction to generate a cDNA sequence from the mRNA transcript; and
c) amplifying a cDNA sequence generated in step (b);
   wherein the reverse-transcription reaction in step (b) is selective for reverse transcription of the mRNA transcript encoding the ABL T315I mutation over a corresponding alternative ABL transcript that does not contain the mutation.

The transcription reaction in this aspect preferably comprises:
(i) annealing a reverse primer to a region of the mRNA transcript comprising a mutation site, wherein the mutation site is responsible for the ABL T315I mutation; and
(ii) extending the reverse primer to generate a cDNA sequence from the mRNA transcript; wherein the mRNA transcript encoding the ABL T315I mutation and the alternative transcript differ in the identity of the base at the mutation site, and wherein selectivity for reverse transcription of the mRNA encoding for the T315I mutation is achieved, at least in part, by the presence of a base in the reverse primer which is complementary to the base at the mutant site of the mRNA but which establishes a mismatch at the base in the corresponding position of the mutation site in the alternative transcript. Preferably, the base is at the 3′ end of the reverse primer.

Step (c) preferably further comprises annealing a forward primer to the cDNA sequence and performing a polymerase chain reaction (PCR) on the cDNA sequence. In a further embodiment, the reverse transcription reaction and PCR reaction employ the same reverse primer. The reverse transcription reaction and PCR reaction may be carried out using the same enzyme, optionally wherein the enzyme is rTth. An example sequence for the reverse primer is a primer comprising the sequence 5′ CCGTAGGTCATGAACTCAA.

It will be appreciated that in this aspect, the ABL T315I mutation may be present on a fusion protein, particularly a BCR-ABL fusion protein. In such a case, the 315^{th} amino acid in the fusion protein may not be the same position as the 315^{th} amino acid in non-fused ABL. However, a skilled person would readily be able to identify the corresponding amino acid position in the fusion protein, since a skilled person skilled in the art can readily align similar sequences and locate the same mutant positions. For example, the amino acid position corresponding to the position of the ABL315 mutation site in the fusion protein sequence recited in SEQ ID NO. 13 is amino acid position 1191. Accordingly, reference herein to the presence of absence of mutant ABL T315I encompasses the detection of the presence or absence of the corresponding mutant in a fusion protein. This applies where e.g. part of the ABL sequence is truncated so that the amino acid number of the mutation position in the fusion protein differs from the amino acid number 315. As described above, a skilled person would have no difficulty in identifying the corresponding position in the fusion protein by simply taking into account any offset (e.g. truncation) that may occur as a result of protein fusion. Where further mutations are recited elsewhere in the present application, the corresponding interpretation of the mutations in the context of their presence in fusion proteins is to be applied accordingly.

Detection of the ABL mutant can be used to identify how the patient will likely respond to administration of a drug, such as tyrosine kinase inhibitor or a BCR-ABL inhibitor e.g. imatinib.

Also provided herein is a method of detecting for the presence of a mutation in the ABL gene coding for the ABL T315I mutation, the method comprising:
a) providing a sample comprising an mRNA transcript;
b) contacting the sample with reagents capable of performing a reverse-transcription reaction when mRNA encoding for the ABL T315I mutation is present, thereby generating a cDNA sequence from the mRNA transcript when mRNA encoding for the ABL T315I mutation is present; and
c) amplifying, if present, a cDNA sequence generated in step (b); wherein the reverse-transcription reaction in step (b) is selective for reverse transcription when the mRNA transcript encoding the ABL T315I mutation is present over a corresponding alternative ABL transcript that does not contain the mutation.

In a further embodiment of this method, the reagents in step (b) comprise a reverse primer which is selective for reverse transcription of the mRNA encoding for the T315I mutation by the presence of a base in the reverse primer which is complementary to the mRNA base responsible for the T315I mutation but which establishes a mis-match in the alternative transcript. The base is preferably at the 3′ end of the reverse primer. The mRNA transcript will typically encodes a BCR-ABL fusion protein or a portion thereof. Where the method of amplification is PCR amplification, the reverse transcription reaction and PCR reaction may employ the same reverse primer. Also provided herein is a method of detecting for the absence of a mutation in the ABL gene coding for the ABL T315I mutation, the method comprising:
a) providing a sample comprising an mRNA transcript of ABL coding for amino acid T at the position corresponding to position 315 of ABL;
b) performing a reverse-transcription reaction to generate a cDNA sequence from the mRNA transcript; and
c) amplifying a cDNA sequence generated in step (b); wherein the reverse-transcription reaction in step (b) is selective for reverse transcription of the mRNA transcript of ABL coding for amino acid T at the position corresponding to position 315 of ABL over a mutant transcript coding for amino acid I at the position corresponding to position 315 of ABL.

In an embodiment of this method, the reverse transcription reaction comprises:
(i) annealing a reverse primer to a region of the mRNA of ABL coding for amino acid T at the position corresponding to position 315 of ABL; and
(ii) extending the reverse primer to generate a cDNA sequence from the mRNA transcript; wherein selectivity for reverse transcription of the mRNA is achieved, at least in part, by the presence of a base which establishes a mis-match in the mRNA transcript encoding the ABL T315I mutation. The base is preferably at the 3′ end of the reverse primer. The mRNA transcript will typically encodes a BCR-ABL fusion protein or a portion thereof. Where the method of amplification is PCR amplification, the reverse transcription reaction and PCR reaction may employ the same reverse primer.

Additionally, the invention described herein may provide valuable information about the quantity of a mutant transcript present in a sample as a means to monitor drug efficacy and disease progression. For example, although there is currently no targeted therapy for patients with a JAK2 V617F mutation there are a number of development efforts underway which target this mutation in patients with Polycythemia Vera. Monitoring the efficacy of a JAK2 inhibitor with a quantitative JAK2 V617F at the transcript level may provide clinically relevant information similar to how BCR-ABL RT-PCR is used for monitoring therapy and disease progression in patients with CML.

It will be appreciated that the present invention can be used to determine or assess any mutant or particular polymorphs is expressed in mRNA. In addition to selecting therapies, or determining diagnosis or prognosis based on mutation or polymorph (e.g. SNP) profile, the present invention may also be used to determine the presence (or absence) of an active gene carrying a mutation by detecting the presence (or absence) of a mutation at the transcript level. This provides an additional level of confidence that the mutated allele is actually driven from an active promoter and therefore producing the targeted protein.

Accordingly, the method of the present invention may be used simply to determine whether the target allele is expressed at the transcript level. Alternatively, or in addition, the present invention may be used to determine the extent to which the target allele is expressed at the transcript level.

Whilst the present invention need not be limited to any particular genes, preferred gene targets include HER2, PI3K, KRAS, EGFR, c-MET, MEK, PTEN, NRAS, HRAS, FGFR1, JAK2, ABL (e.g. ABL T315I), EGFR (e.g. EGFR T790M and/or L858R), MEK, EGFR, BRAF (e.g. BRAF V600E, BRAF V600D, BRAF V600R, BRAF V600K) and ALK. The present invention also provides a kit containing reagents for performing the method of the present invention. The kit may also contain instructions for performing the method of the present invention. For example, the kit may contain reagents and/or instructions for selectively producing and amplifying a cDNA sequence of a target allele of a gene by reverse transcription PCR, wherein the target allele is a mutant allele or is a specific allele of a polymorphic gene, and wherein the kit comprises:
(i) a reverse primer specific to a region of an mRNA transcript of the target allele comprising a target site, wherein the mRNA transcript of the target allele of the gene and the mRNA of an alternative allele of the gene differ in base composition at the position of the target site, and wherein the reverse primer comprises one or more bases which are complementary to the mRNA sequence at the target site of the target allele but which establish a mis-match at the position of the target site in the alternative allele;
(ii) a forward primer specific for an upstream region of the mRNA transcript of the target allele;
(iii) a reverse transcriptase; and
(iv) a DNA polymerase.

In a preferred embodiment of this aspect, the selectivity for reverse transcription of the target allele mRNA over the alternative allele mRNA is achieved, at least in part, by a base at the 3′ end of the reverse primer which establishes a mis-match with the mRNA sequence of the alternative allele. Preferably, the reverse transcriptase and the DNA polymerase is the same enzyme. In a further preferred embodiment, the enzyme is rTth polymerase. The kit may contain further reagents, for example, an oligonucleotide probe that allows detection of the amplification product using real-time PCR. Such a probe may contain a sequence that hybridises to the amplified cDNA and contains a fluorescent dye (the reporter) attached to one end of the oligonucleotide and a quencher, which absorbs light emitted from the reporter when in close proximity to it, is bound to the other end. The kit may also comprise deoxynucleoside triphosphates (dNTPs).

In a further embodiment, the present invention provides a kit for detecting the presence of a mutation in the ABL gene coding for the ABL T315I mutation, wherein the kit comprises: (i) a reverse primer specific to a region of an mRNA transcript encoding the ABL T315I mutation, and wherein the reverse primer comprises a base which is complementary to the mRNA base responsible for the T315I mutation but which establishes a mismatch in the corresponding base of the wild type mRNA lacking the mutation; (ii) a forward primer specific for an upstream region of the mRNA transcript; (iii) a reverse transcriptase; and (iv) a DNA polymerase.

The present invention will now be described with reference to the following non-limiting examples.

### EXAMPLES

### Materials and Methods

### Cell Lines

Equal amounts of RKO cells were distributed into ten different 1.7 ml snap top tubes. Total RNA was isolated from five of the tubes using the Stratagene RNA Isolation kit and DNA was isolated from the second set of five using the Qiagen DNA Mini kit. The nucleic acid yields were assessed using a NanoDrop 1000. The concentrations were not normalized between the various samples in an attempt to maintain cell equivalency between each tube.

### Tissue Sections

Formalin-fixed paraffin embedded tissue sections with known mutational status were cut at 5 uM and mounted on glass slides. H&Es were prepared from one section to verify tumor content and the others were used for DNA and RNA isolation. The whole sections were removed from the slides and incubated in digestion buffer to release the cellular content. DNA and RNA were isolated from the cell lysate using a column based extraction protocol optimized for fixed and embedded tissues. A NanoDrop 1000 was used to assess the quantity and quality of the nucleic acids. As with the cell line studies the concentrations were not normalized in an attempt to maintain an equal number of target cells for each extraction.

BRAF assay

### Allele Specific RT-PCR

An allele specific PCR was developed and optimized for the wild-type sequence as well as the V600E mutation of BRAF. The assay was designed to target either the nucleotide of the wild-type sequence or the V600E mutated sequence at the terminal 3′ nucleotide of the reverse primer. Both the wild-type and mutation specific primer sets were identical with the exception of the terminal 3′ position of the reverse primer. A series of primers were evaluated for analytical sensitivity as well amplification specificity for each sequence. The primers with the optimal performance were selected for further studies. The primer sequences are identified in below.

The DNA-based PCR was performed in 10 ul volumes using Applied Biosystems Fast Advanced master mix. The master mix contains all components required to perform PCR in a predesigned formula with the exception of the assay specific primers. The same primer/probe set was found to be optimal for both the DNA and mRNA based reactions and was added to the reaction at 0.4 uM for each primer and 0.2 uM for the probe. The probe was an MGB probe labelled with a FAM reporter. Two ul of the DNA preparations were subjected to 40 amplification cycles using an Applied Biosystems 7900 HT. The cycling parameters were 50° C. for 2 minutes, 95° C. for 20 seconds, followed by 40 cycles of 95° C. for 1 second and 60° C. for 20 seconds.

The RNA based reactions were also performed in 10 ul but used EZ One Step Chemistry from Applied Biosystems. The EZ One Step Chemistry has all components required to perform one-step RT-PCR with the exception of the gene specific primers and probe. As previously mentioned the primer/probe set used for the RT-PCR was the same as was used in the DNA based assay. The optimal primer/probe concentration for the RT-PCR was also 0.4 uM for each primer and 0.2 uM for the probe. As with the DNA based assay 2 ul of the RNA preparation were subjected to an initial hold 60° C. for 30 minutes for the conversion to cDNA followed, followed by 40 cycles of 95° C. for 15 second and 60° C. for 60 seconds.

BRAF Gene segment (SEQ ID NO: 1)
[SEQ ID NO: 1]
5′TAATATATTTCTTCATGAAGACCTCACAGTAAAAATAGGTGATTTTGG
TCTAGCTACAGTGAAATCTCGATGGAGTGGGTCCCATCAGTTTGAACAGT
TGTCTGGATCCATTTTGTGGATGGCACCAGAA

In SEQ ID NO:1, the exon boundaries of exon 15 are defined by the nucleotides at positions 4 and 121. In the V600E mutant the T base at position 60 of SEQ ID NO: 1 is replaced by A (shown by the underscore).

Reverse primer for wild-type (BRAF RPWT)
(SEQ ID NO: 2)
5′CAC TCC ATC GAG ATT TCA
Tm: 48.2% GC: 44 18 bp
Reverse Primer for mutant (BRAF RPMUT))
(SEQ ID NO: 3)
5′C CAC TCC ATC GAG ATT TCT
Tm: 51.1% GC: 47 19 bp
Forward Primer (BRAF FP, in same exon)
(SEQ ID NO: 4)
5′ATA TTT CTT CAT GAA GAC CTC ACA GTA
Tm: 55.7% GC:33 27 bp
Real-time PCT Probe (BRAF Probe)
(SEQ ID NO: 5)
5′AGG TGA TTT TGG TCT AGC TAC
Tm: 70.0% GC: 43 21 bp

The FP/RPWT (Wild-type) primer combination generates a 72 bp product

The FP/RPMUT (Mutation) primer combination generates a 73 bp long product.

The FP/RPWT primer combination is used as an amplification control to verify the presence of amplifiable BRAF sequence in the sample.

### Results

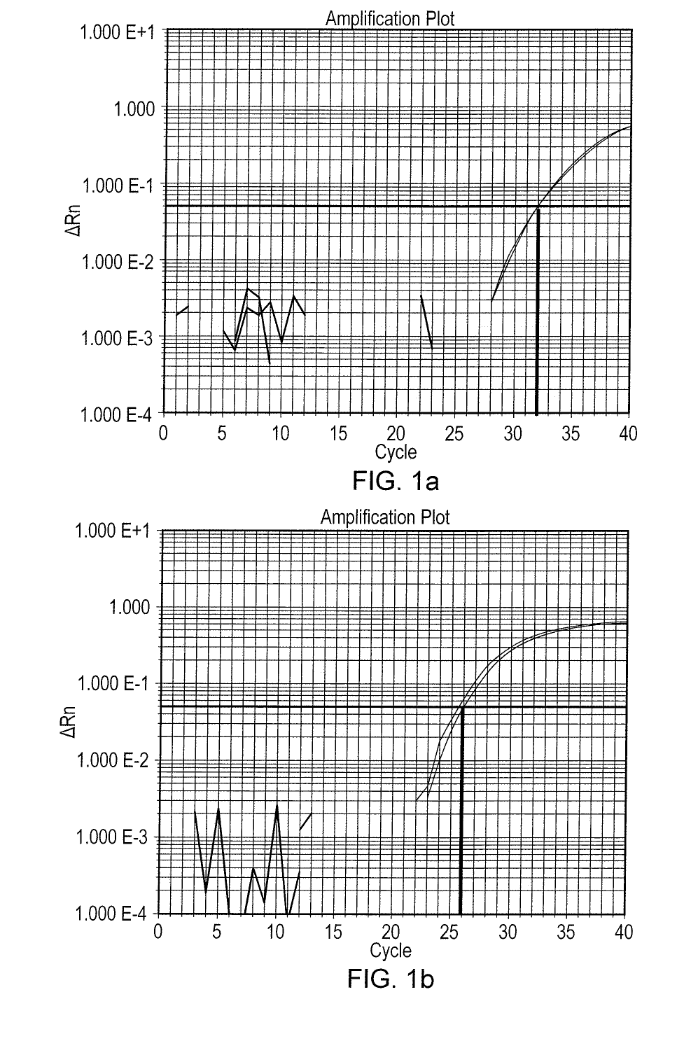

The BRAF V600E One-Step Allele Specific RT-PCR was capable of detecting the mutant transcript in each sample that was tested with a known mutation. FIG. 1a is an amplification plot generated from a DNA based AS-PCR while FIG. 1b demonstrates an amplification plot generated from the RNA fraction of the same sample using the AS-RT-PCR methodology. Both fractions were isolated from the same number of cells collected from a tumour biopsy known to harbour the BRAF V600E mutation. FIGS. 1a and 1b show amplification plots with Ct values of 31.8 and 25.8, respectively. The difference in Ct values generated between the two methodologies (delta Ct) is approximately 6 cycles for this sample. A difference of 6 cycles translates to a 64 fold increase in sensitivity achieved with the RNA based assay over the traditional DNA based AS-PCR.

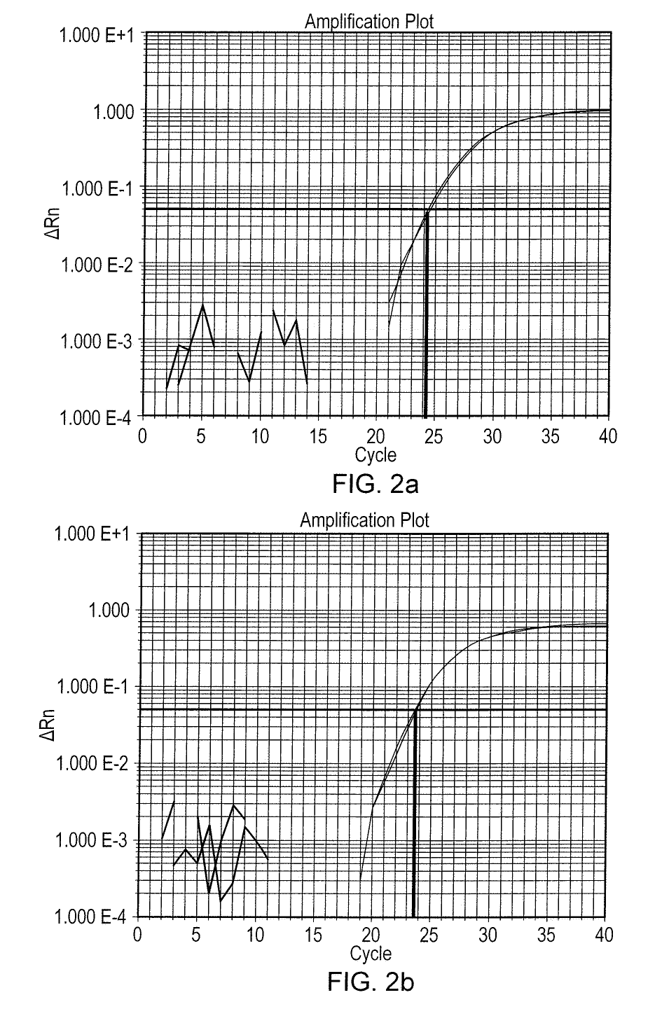

FIGS. 2a and 2b show results generated from the same sample where the AS-RT-PCR provided a more modest advantage over the DNA based assay. The Ct values generated from the DNA and RNA based assays were 24.4 and 23.6, respectively. The delta Ct for this sample was 0.8 Ct which calculates to about a 1.7 fold increase in sensitivity. Although the delta Ct value was low, the RNA based assay still provided a sensitivity advantage over the DNA based approach.

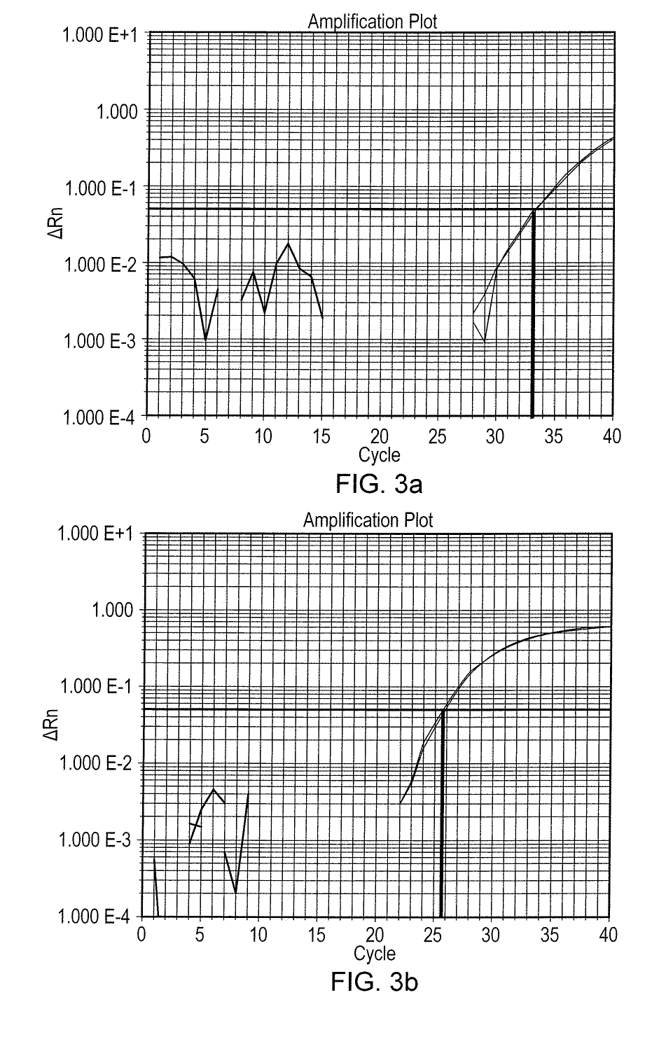

FIGS. 3a and 3b also demonstrate variable sensitivities between the DNA and RNA based methodologies. In this sample the DNA based assay generated a Ct of 33.2 and the RNA based approach crossed the threshold at 25.7. The delta Ct for this sample is 7.5 which translates to a 181 fold difference in sensitivity between the two methods.

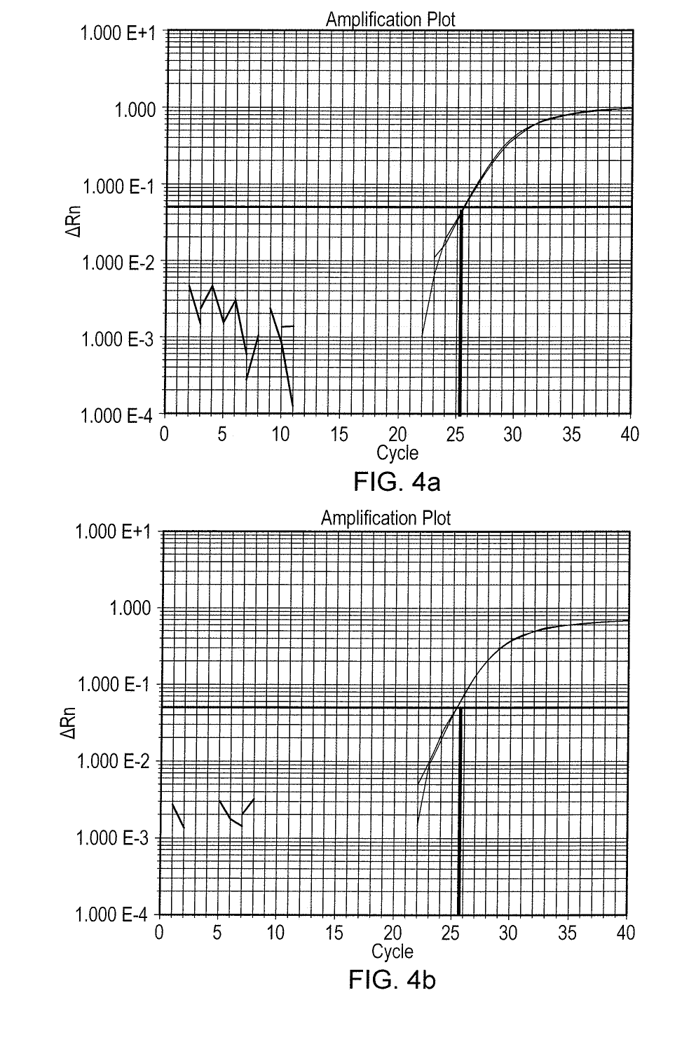

FIGS. 4a and 4b demonstrate a sample that likely had an equivalent number of mutated DNA copies and mRNA transcripts per cell. The Ct values generated from each fraction are almost identical indicating a close to equal number of targets per fraction. The DNA based assay had a Ct of 25.4 while the RNA AS-RT-PCR generated a Ct of 25.3.

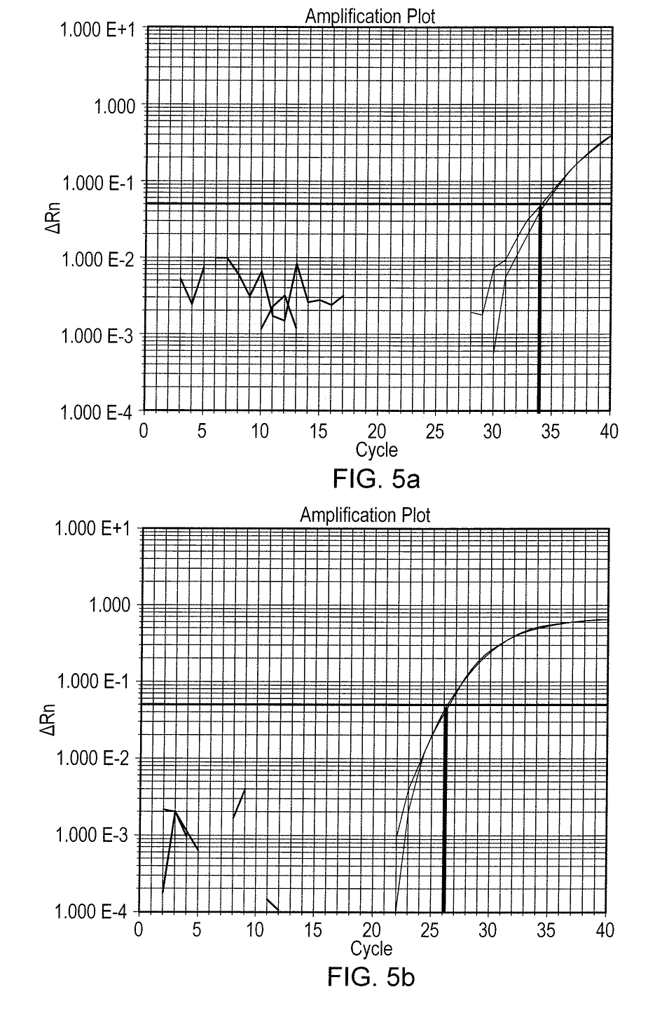

FIGS. 5a and 5b were generated from a sample that showed a significant enhancement in sensitivity from the RNA based assay. The Ct value from the traditional AS-PCR is 34 while the AS-RT-PCR is 26.3. The delta Ct for this sample is 7.7 cycles between each fraction which is a 208 fold increase in sensitivity from the AS-RT-PCR methodology. Although some samples generated similar Ct vales for the DNA and RNA fractions, on no occasion was a higher Ct value observed for the mRNA fraction when compared to results generated using DNA for any sample set that was tested. Additionally we were able to generate results from samples that were either QNS for DNA based assays or failed to generate a result when tested.

### ABL Assay

Allele specific PCR was developed for the T315I mutation. The assay was developed and optimized for the wild-type reaction as well as the mutation. The T315I mutation is a result of a C>T point mutation at the 947 nucleotide of the ABL gene. The reverse primers of the assay had either a C or T at the last base at the 3′ position to selectively amplify the wild-type or mutated sequence. Primers (tablet) were screened for analytic sensitivity, reaction efficiency and amplification specificity using a RNA extracted from a cell line homozygous for the T315I mutation and K562 RNA (Promega). The reactions were performed on an ABI 7900HT using EZ RT-PCR Core Reagents (Applied Biosystems) which contained all the necessary components with the exception of the gene specific primers and probe. Two ul of the RNA fraction were subjected to a 60° C. hold for 30 minutes, 95° C. for 15 seconds, followed by 40 cycles of 95° C. for 15 second and 60° C. for 60 seconds. EZ One Step chemistry was chosen to take advantage of the higher reverse transcription temperature associated with rTth polymerase.

TABLE 1
T315I Primers and Probe

| Name | Direction | Sequence |
|---|---|---|
| T315I F | Sense | 5′ CTGGTGCAGCTCCTTGGG |
| | | (SEQ ID NO: 6) |
| T315I RWT | Antisense | 5′ CCGTAGGTCATGAACTCAG |
| | | (SEQ ID NO: 7) |
| T315I RMut | Antisense | 5′ CCGTAGGTCATGAACTCAA |
| | | (SEQ ID NO: 8) |
| T315I Probe | Sense | FAM-5′ CCCCGTTCTATATCATC |
| | | (SEQ ID NO: 9) |

*Underscored base represents nucleotide targeted in each reaction.

While the particular embodiment of the present invention has been shown and described, it will be obvious to those skilled in the art that changes and modifications may be made without departing from the teachings of the invention. The matter set forth in the foregoing description and accompanying drawings is offered by way of illustration only and not as a limitation. The actual scope of the invention is intended to be defined in the following claims when viewed in their proper perspective based on the prior art.

What is claimed is:

1. A method of selectively producing and amplifying a cDNA sequence of a target allele of a gene, wherein the target allele is a mutant allele or is a specific allele of a polymorphic gene, the method comprising:
a) providing a sample comprising an mRNA transcript of the target allele;
b) performing a reverse-transcription reaction to generate a cDNA sequence from the mRNA transcript of the target allele; and
c) amplifying a cDNA sequence of the target allele generated in step (b);
wherein the reverse-transcription reaction in step (b) is selective for reverse transcription of the mRNA transcript of the target allele over an mRNA transcript of an alternative allele of the same gene.

2. The method of claim 1, wherein the target allele is a mutant allele and the alternative allele is the wild-type allele.

3. The method of claim 1, wherein the target allele is a specific allele of a polymorphic gene comprising a polymorphic site, and the target allele and alternative allele differ in base composition at the polymorphic site.

4. The method of claim 3, wherein the polymorphic site is a single nucleotide polymorphism (SNP) site.

5. The method of claim 1, wherein the reverse-transcription reaction comprises:
(i) annealing a reverse primer to a region of the mRNA transcript of the target allele comprising a target site; and
(ii) extending the reverse primer to generate a cDNA sequence from the mRNA transcript of the target allele;
wherein the mRNA transcript of the target allele and the mRNA of the alternative allele differ in base composition at the position of the target site, and wherein selectivity for reverse transcription of the target allele mRNA over the alternative allele mRNA is achieved by the presence of one or more bases in the reverse primer which are complementary to the mRNA sequence at the target site of the target allele but which establish a mismatch at the position of the target site in the alternative allele.

6. The method of claim 5, wherein the target site is a mutation site or a SNP site.

7. The method of claim 5, wherein the reverse primer binds with full complementarity to the mRNA of the target allele.

8. The method of claim 5, wherein the selectivity for reverse transcription of the target allele mRNA over the alternative allele mRNA is achieved, at least in part, by a base at the 3′ end of the reverse primer which establishes a mismatch with the mRNA sequence of the alternative allele.

9. The method of claim 5, wherein the reverse primer is between 10 and 30 nucleotides in length.

10. The method of claim 5, wherein step (c) comprises annealing a forward primer to the cDNA sequence and performing a polymerase chain reaction (PCR) on the cDNA sequence.

11. The method of claim 10, wherein the reverse transcription reaction and PCR reaction employ the same reverse primer.

12. The method of claim 11, wherein the forward primer and reverse primer are specific for the same exon of the target allele.

13. The method of claim 10, wherein the reverse transcription reaction and PCR reaction are carried out using the same enzyme, optionally wherein the enzyme is rTth.

14. The method of claim 1, wherein the sample is substantially free of DNA.

15. The method of claim 1, wherein the target allele is the mutant allele of the human BRAF gene encoding a V600 mutation, and the method is selective for producing and amplifying cDNA of the V600 mutation over cDNA of wild-type BRAF.

16. The method of claim 1, further comprising detecting and/or quantifying the presence of the amplified cDNA.

17. The method of claim 16, wherein the amplified cDNA is detected by real-time PCR.

18. The method of claim 1, wherein the target allele is an allele of HER2, PI3K, KRAS, EGFR, c-MET, MEK, PTEN, NRAS, HRAS, FGFR1, JAK2, EGFR, MEK, EGFR or ALK.

19. The method of claim 1, wherein the target allele is BRAF V600E, BRAF V600D, BRAF V600R, BRAF V600K, EGFR L858R, EGFR T790M, or ALK C1156Y.

20. The method of claim 1, wherein the presence of the target allele is predictive of a diagnosis and/or a prognosis of a subject from which the sample is taken.

21. The method of claim 20, further comprising detecting the amplified cDNA of the target allele and assessing from the detection of the amplified cDNA a diagnosis and/or a prognosis of the subject.

22. The method of claim 1, wherein the sample is from a subject known to have, or suspected to have, a disease, and wherein the presence of the target allele is predictive of how the subject will respond to administration of a drug to treat the disease.

23. The method of claim 22, further comprising detecting the amplified cDNA of the target allele and assessing from the detection of the amplified cDNA the likelihood of success of treating the subject with the drug.

24. The method of claim 22, wherein the target allele is a mutant allele of the human BRAF gene encoding the V600 mutation and the drug is vemurafenib.

25. A kit for selectively producing and amplifying a cDNA sequence of a target allele of a gene by reverse transcription PCR, wherein the target allele is a mutant allele or is a specific allele of a polymorphic gene, and wherein the kit comprises:
(i) a reverse primer specific to a region of an mRNA transcript of the target allele comprising a target site, wherein the mRNA transcript of the target allele of the gene and the mRNA of an alternative allele of the gene differ in base composition at the position of the target site, and wherein the reverse primer comprises one or more bases which are complementary to the mRNA sequence at the target site of the target allele but which establish a mis-match at the position of the target site in the alternative allele;
(ii) a forward primer specific for an upstream region of the target allele;
(iii) a reverse transcriptase; and
(iv) a DNA polymerase.

26. The kit of claim 25, wherein the selectivity for reverse transcription of the target allele mRNA over the alternative allele mRNA is achieved, at least in part, by a base at the 3′ end of the reverse primer which establishes a mismatch with the mRNA sequence of the alternative allele.

27. The kit of claim 25, wherein the reverse transcriptase and the DNA polymerase are the same enzyme.

28. A method of detecting for the presence of a gene mutation, the method comprising:
a) providing a sample comprising an mRNA transcript;
b) contacting the sample with reagents capable of performing a reverse-transcription reaction when mRNA containing the mutation is present, thereby generating a cDNA sequence from the mRNA transcript when mRNA encoding for the mutation is present; and
c) amplifying, if present, a cDNA sequence generated in step (b);
wherein the reverse-transcription reaction in step (b) is selective for reverse transcription when the mRNA transcript containing the mutation is present over the alternative transcript of the gene that does not contain the mutation.

29. The method of claim 28, wherein the reagents in step (b) comprise a reverse primer which is selective for reverse transcription of the mRNA containing the mutation by the presence of a base in the reverse primer which is complementary to the mRNA base containing the mutation but which establishes a mismatch in the alternative transcript.

30. The method of claim 29, wherein the base is at the 3′ end of the reverse primer.

31. The method of claim 29, where step (c) comprises annealing a forward primer to the cDNA sequence and performing a polymerase chain reaction (PCR) on the cDNA sequence.

32. The method of claim 31, wherein the reverse transcription reaction and PCR reaction employ the same reverse primer.

33. The method of claim 28, wherein the mutation is in HER2, PI3K (PIK3CA), KRAS, EGFR, c-MET, MEK, PTEN, NRAS, HRAS, FGFR1, JAK2, BRAF or ALK.

34. The method according to claim 1, wherein the mRNA transcript of the alternative allele is not present in the sample.
